SEQUENCE LISTING (12 sequences)

SEQ ID NO: 1 - protein, 334 aa, Yarrowia lipolytica
MKLSTILFTACATLAAALPSPITPSEAAVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNGHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 2 - DNA, 1005 bp, Yarrowia lipolytica
atgaagctttccaccatcctcttcacagcctgcgctaccctggctgccgccctcccttcccccatcactccttctgaggccgcagttctccagaagcgagtgtacacctctaccgagacctctcacattgaccaggagtcctacaacttctttgagaagtacgcccgactcgcaaacattggatattgtgttggtcccggcactaagatcttcaagcccttcaactgtggcctgcaatgtgcccacttccccaacgttgagctcatcgaggagttccacgacccccgtctcatctttgatgtttctggttacctcgctgttgatcatgcctccaagcagatctaccttgttattcgaggaacccactctctggaggacgtcataaccgacatccgaatcatgcaggctcctctgacgaactttgatcttgctgctaacatctcttctactgctacttgtgatgactgtcttgtccacaatggcttcatccagtcctacaacaacacctacaatcagatcggccccaagctcgactctgtgattgagcagtatcccgactaccagattgctgtcaccggtcactctctcggaggagctgcagcccttctgttcggaatcaacctcaaggttaacggccacgatcccctcgttgttactcttggtcagcccattgtcggtaacgctggctttgctaactgggtcgataaactcttctttggccaggagaaccccgatgtctccaaggtgtccaaagaccgaaagctctaccgaatcacccaccgaggagatatcgtccctcaagtgcccttctgggacggttaccagcactgctctggtgaggtctttattgactggcccctgatccaccctcctctctccaacgttgtcatgtgccagggccagagcaataaacagtgctctgccggtaacactctgctccagcaggtcaatgtgattggaaaccatctgcagtacttcgtcaccgagggtgtctgtggtatctaa

SEQ ID NO: 3 - protein, 498 aa, Yarrowia lipolytica
MPLELPSLNASIVGNTVQNGAVEQFLNIRYADIPGKFEKPVLKNDWNGAEIDATKVGPVCPQPRTPFNFFSVPDDLWEKVNVDTYQDGLLCDNLIVTRPKGVSANARLPTVVWIHGGSNIEGSIYNLIYEPQFLVAESVRVGKPIVHVCIEYRLGLAGFLTKNGKGNWGTWDQYTGCQWVNRHIQDFGGDPLNVTLTGESAGSVAVHNMLIKDSMNGRKLFRNAVMMSGTLETITPQPPKWHARLEEKVAKVTGKEVADLASLSDKELLDAQIKLNVAVCMTCDDGDFFEPGWKQHLTPDWLDKLIISDCKDEGMLYFLPVNAQDDEELLAKVAKSPVGKEISELYGIKEGGDIKSACLDLKTDATFNYFNHLLFKKMEEARNNGSTSRVYRLAVDEPNPHNPDQRAHHAVDVLYMFNSTKFNEHGDKLSRLFQSHFLRLAYGLEPWDHRNFGVYRNGGYQQLPLSELNKVRPVERYEALSKMDFGQVGRLSNALSRL

SEQ ID NO: 4 - DNA, 1497 bp, Yarrowia lipolytica
atgcctctcgaactcccctcgctcaacgcctcgattgtcggcaacaccgttcagaacggcgctgttgagcagtttctcaacatccgatacgccgacattcctggcaagtttgagaagcccgtgctcaagaacgattggaacggcgcggagatcgacgccaccaaggtcggtcccgtgtgcccccaaccccgcaccccattcaacttcttctccgtgccagacgacctctgggagaaagtcaatgtggacacgtaccaggacggtctgctgtgcgacaacctgattgtgacgcgaccgaagggcgtgtctgccaacgcccggctgcccactgttgtgtggatccacggcggctccaatattgagggcagtatctacaacctcatctatgagccccagttcctggtggcagagtcggtgcgagtaggcaagccgattgtgcacgtgtgtatcgagtaccgactgggtctcgcgggcttcctcaccaagaacggcaagggcaactggggcacgtgggatcagtacacgggctgccagtgggtcaaccgccacattcaggactttggaggcgatcctttgaacgtgacattgaccggtgagtctgccggctctgtagcagtccataacatgctcatcaaggactccatgaacggccgaaagttgttccgaaatgccgtcatgatgagtggcactctcgagaccatcactcctcagcctcccaagtggcatgctcgtttggaggagaaggtggccaaggtcactggcaaggaagtggccgaccttgcttctctgtccgataaggagctgctcgacgcccagatcaagctcaatgtggctgtgtgcatgacttgcgacgacggcgactttttcgagcccggatggaagcagcatctgactcctgactggctcgacaagctcatcatctccgattgcaaggacgagggcatgctgtatttcctgccagtcaacgcgcaggacgacgaggagctgttggcaaaggtggccaagtcgcccgtgggtaaggagatttccgagctttacggcatcaaggagggtggcgatatcaagtctgcgtgtctcgatctcaagactgacgccaccttcaattactttaaccatctgctgttcaagaagatggaggaggcccgaaacaacggctccacttctcgagtttaccgtctggccgtcgatgagcccaacccccacaaccccgaccagcgggcccaccacgccgtcgacgtgctgtacatgttcaactcgaccaagttcaacgagcacggcgacaagctgtctcggctgttccagagccactttttgcggctggcgtatggcctggagccctgggaccatcgaaactttggagtgtacagaaacggcggctaccagcagctgccgctgagtgagttgaacaaggtccgacccgtcgagcggtacgaggcgctgtccaagatggactttggccaggttgggcgtttgtccaatgcgctttcgcgcctatga

SEQ ID NO: 5 - protein, 371 aa, Yarrowia lipolytica
MVSLSARIKDFFSVLLLGAATITPSTQTAGVSQGFYDFARDFAHLSNIAYCVNAPITPLNPDFTCGNSCKHFPEIELVKTFGGNFFKTSITGYLAVDHVKKEKYVVFRGTFSLADAITDMQFQLSPFLVDVPALNTFSANDTTAEAQTHCEGCKIHDGFSKAFTETWGNIGEDLQKHLDANPDYQLYVTGHSLGAAMALLGATSIKLKGYDPILINYGQPRVGNKPFAEFINKLWFGEGNGLEITPERKLYRMTHWNDIFVGLPNWEGYTHSNGEVYINNRFINPPLKDVISCAGGENSKCYRSSFSLLSQINLLQNHLAYIDYIGYCALNIGRRELADQEHYTGPYYYGHRSEEDFKKLGLELSTPQVEN

SEQ ID NO: 6 - DNA, 1116 bp, Yarrowia lipolytica
atggtatccctctctgctcgaatcaaagactttttttcggtcctcctcctcggagctgcaaccatcactccctccacacagaccgcaggcgtgtctcaagggttctatgattttgctcgggactttgcccatctgtccaacattgcctactgtgtcaatgctcccatcactccactgaacccggacttcacctgtggcaactcgtgcaagcactttccggaaattgagcttgtgaagacatttggaggcaacttcttcaagacctccattacgggctacctggctgtcgatcatgtcaagaaggagaagtacgttgtcttccgaggaaccttttcgctggcagacgcgatcacggacatgcagttccagctgtctcctttcctggtcgatgtgcctgccctgaacactttctcagctaatgacaccaccgcagaggcccagacgcactgtgagggctgcaaaattcacgacggcttctccaaggcctttaccgagacctggggtaacattggtgaggatctgcagaaacacctggacgctaacccggactaccagctgtacgtgactggccattctctgggagctgctatggcccttcttggagctacttccatcaagctcaagggctacgatcccattctcatcaactacggacagccccgagtcggaaacaagcccttcgctgagttcattaacaagttgtggtttggagaaggcaacggtctggaaatcacccccgagagaaagctgtaccgaatgacccactggaacgacatctttgttggcctgcccaactgggagggatacacccactctaacggtgaagtatacatcaacaaccggttcatcaaccctcctctcaaggatgtcatctcttgtgctggaggcgaaaactcgaagtgctaccgatcctcgttcagcctgctgtcccagatcaatctgctccaaaaccacctggcttacattgattacattggatactgcgctctgaacattggtcgacgagagcttgccgatcaggaacattacactggtccttattactatggtcatcgatctgaggaggactttaagaagttgggcttggagctatccaccccacaagttgagaactga

SEQ ID NO: 7 - protein, 406 aa, Yarrowia lipolytica
MAGFNFTFGQVISYLASMLYGQVDATSSSTRIQATQDLYDFTAKFSRLSNIAYCINAPFTPLRTDFTCGESCRYFPDLQLDSVFGGNFSSASTTGYIAYDHKKKEKYIVFRGTFSIPDIITDIQFQTAPWLTSLPTHLIPTKEDFEHKQAILKHYAAENKGLSNLEERQDVVHEDPSLVPKKMDKCENCQIHDGFAKGFNETIEHAGPQIEKFLGNNTDYKMFVVGHSLGAAQAQLFATQFKLLGFDPYMINFGQPRLGNPEFAAYINQLWFNDTGLVVNDARRFYRVTHWNDIVVGVPDWLNYTHSIGEVFIDEESVYPKLDKVVVCEGGENPLCHRGTFNLWSRINFLQNHLAYIFYIGLCAFNIGRRDVLNMPQYQGNFSYQHNIDPNYNYDTKVPTRISKSN

SEQ ID NO: 8 - DNA, 1221 bp, Yarrowia lipolytica
atggcagggttcaacttcactttcgggcaggtgatttcgtacctggcttccatgctctatggacaggtggatgccacttcatcctccactcgaatccaggccacccaggatctgtacgacttcacagccaagttttcgcgactctcaaacatcgcgtactgcatcaatgcccccttcacgcctctcagaacggacttcacctgcggagaaagctgtcggtacttccccgacctccagctggactcagtgtttggtggtaacttctcctcagcctccactaccggctacattgcatacgaccacaagaagaaggaaaagtacattgtgtttcgaggaactttcagtatccctgatatcatcacagacattcaatttcagactgccccttggttgacctctctgcccacgcatctgatccctaccaaggaggactttgaacacaagcaggccatcctgaagcactacgctgccgaaaacaagggtctcagcaacctggaagagcgacaggatgttgtgcatgaagaccctagcctggttcccaagaaaatggacaagtgcgagaactgccagatccatgacggattcgccaagggcttcaacgagactatcgagcatgccggaccccagattgaaaagttcctgggcaataacaccgactacaagatgtttgttgttggccactctctaggagctgctcaggcccagctgtttgctacacagttcaaactgctgggatttgacccttacatgatcaactttggacagcctcgacttggaaaccctgagttcgccgcctacatcaaccagctgtggttcaacgatactggtctggttgtcaatgatgcccgacgattctaccgagtgactcactggaacgatatcgtcgtgggagtgcccgactggctcaattacacccactctatcggagaggtgttcatagacgaggagagcgtttaccccaagctggacaaggtggtggtgtgcgagggaggagagaaccccctgtgccaccgaggaactttcaacctgtggtcacgaatcaacttcctgcagaaccatttggcttatatcttctacattggtctgtgtgctttcaacattggccgaagagacgtgctcaacatgccacaataccagggcaacttctcgtaccagcacaacatcgaccccaactacaattacgataccaaggttcccacccggatcagtaaatcaaactaa

SEQ ID NO: 9 - DNA, 11606 bp, Artificial Sequence
  plasmid MB7452 expressing Cas9
cgcgtggatcgccggtgcgttgacgttggtgacctccagccagaggtgcccggcgccccgctcgccggcgaactccgtcgcgagccccatcaacgcgcgcccgaccccgtgcccccggtgctccggggcgacctcgatgtcctcgacggtcagccggcggttccacgccgagtacgagatgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgccgctcgccgtccgaggcggtgccgtcgtacagggcggtgtccaggccgcagagggtgaaccccatccgccggta

SEQ ID NO: 10 - DNA, 14006 bp, Artificial Sequence
  plasmid MB9523 expressing DrBCO, LmATF-mutated, FfRDH
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 11 - DNA, 13246 bp, Artificial Sequence
  plasmid MB8388 expressing Cas9, Lip8 targeting guide RNAs
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgacgagactgatgagtccgtgaggacgaaacgagtaagctcgtcgagggctgcaaaattcacgagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtacagcaggctgaacgaggatgttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 12 - protein, 559 aa, Lachancea mirantina
MGDLDARGTSAHPELSERPSIMPSMSDIQDPSGDDKATPRGSAAGLPQLELAGHARRLGHLENFFAVQHRQQIYSSFAVFCEFDTACSLAQLASAVRNVCLSNPLLLHTVEPKHPDIAGFYHSDEYLSRPWPQHDYMRVLREVHVADVVMNGQKEHAHVVRDAVDVFQAHGNQVTSELLELMTQIEIPHASQTRPSWRLLCFPHGEANRWRTFAFVSNHCSSDGLSGLNFFRDLQKELAHGPTSGAPGAPGASGVIFDYAQDAATLPKLPPPIDQKLDYRPSKKALLGLLAGKFVREKLGYVSAAPPTTPTSDLAHPEGHQYYCYLVNVPTSSVAHIKTQVRENVPHKCTLTPFLQACWLVSLFKYGRVFSGSWLERYTDVLVAMNTRQLLPEDLELQRQYRYGSNVGGVRYNYPIAPLDVRDNDQKFWSLVESYRLALSDARDKNDYLYALGALMLPEIYEKKNVDAVVNDTILNQRRSGTLISNVGYVRDEQPTAFAIKNHVFSQGVGANRNAFVLNICATDQGGLNIAISIAKGTLASRQEGQELCDIFKSTLLRF

(19) United States
(12) Patent Application Publication
(10) Pub. No.: US 2024/0018564 A1
(43) Pub. Date: Jan. 18, 2024

(54) IN SITU TWO-PHASE EXTRACTION SYSTEM

(71) Applicant: DSM IP ASSETS B.V., Heerlen (NL)

(72) Inventors: Peter Louis HOUSTON, Kaiseraugst (CH); Valmik Kanubhai VYAS, Kaiseraugst (CH)

(21) Appl. No.: 18/251,167

(22) PCT Filed: Nov. 1, 2021
(86) PCT No.: PCT/EP2021/080283

§ 371 Date: Apr. 28, 2023

Related U.S. Application Data
(60) Provisional application No. 63/107,719, filed on Oct. 30, 2020.

(51) Int. Cl. C12P 23/00; C12N 9/20; C12N 9/10

(52) U.S. Cl. CPC C12P 23/00; C12N 9/20; C12Y 301/01003; C12Y 203/01084; C12N 9/1029

(57) ABSTRACT

The present invention is related to a fermentative production of retinoids, including retinol or retinyl acetate, comprising cultivation of a retinoid-producing host cell, such as fungal host cell, particularly oleaginous host such as e.g. Yarrowia, in a two-phase culture system in the presence of vegetable oil as second phase. The retinoids present in the vegetable oil can be used without further isolation or purification steps as active ingredients in the food, feed, pharma or cosmetic industry.

The present invention is related to fermentative production of retinoids, including retinol or retinyl acetate, comprising cultivation of a retinoid-producing host cell, such as fungal host cell, particularly oleaginous host such as e.g. Yarrowia, in a two-phase culture system in the presence of vegetable oil as second phase. The vegetable oil comprising the retinoids can be directly used without further isolation and/or purification steps as ingredient in the food, feed, pharma or cosmetic industry.

Retinoids, including vitamin A, are one of very important and indispensable nutrient factors for human beings which must be supplied via nutrition. Retinoids promote well-being of humans, inter alia in respect of vision, the immune system and growth. Retinyl acetate is an important intermediate or precursor in the process of vitamin A production.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, comprising microbial conversion steps have been investigated, which would lead to more economical as well as ecological vitamin A production.

In general, the biological systems that produce retinoids are industrially intractable and/or produce the compounds at such low levels that commercial scale isolation is not practical. The most limiting factors include instability of intermediates in such biological systems and/or the relatively high production of by-products, such as e.g. fatty acid retinyl esters, particularly using oleaginous host cells grown on triglycerides as carbon source.

In order to circumvent some of these issues in fermentative production of retinoids, so-called two-phase cultivation systems have been developed, wherein the fermentation products are collected outside the cell in so-called second phase lipophilic solvents such as e.g. Drakeol®, silicone oil or n-dodecane (see WO2020141168 or Jang et al., Microbial Cell Factories 10:59, 2011).

Unfortunately, these solvents are based on synthetic or petroleum compounds and therefore should be avoided due to ecological, economical, health and safety issues.

Thus, it is an ongoing task to look for more eco-friendly and sustainable biological vitamin A production processes, wherein the fermentation products secreted or accumulated outside the host cell are extracted in situ from the host cell by more “natural” solvents, including the use of plant-based second-phase solvents.

Surprisingly, we now found a novel process for in situ extraction of fermentative produced retinoids, including retinol or retinyl acetate, particularly with a fungal host cell, such as e.g. an oleaginous yeast, particularly Yarrowia, said host cell being cultivated in the presence of a plant-based second phase, e.g. vegetable oil, such as e.g. corn oil, as second phase solvent. Using such process with a host cell growing on e.g. glucose or ethanol, the accumulation of total retinoids could be increased in the range of at least about 10% compared to a process using silicone oil as second phase.

As used herein, the term “solvent comprising vegetable oil” or “solvent comprising silicone oil” means that the percentage of vegetable oil and silicone oil, respectively, is at least in the range of about 90%, preferably in the range of about 95, 98, 99, or 100% (v/v).

As used herein, the term “secreted” means the movement of the molecules by mass action or diffusion from the lipids in the cell to the lipid in the second phase.

Suitable plant-based second phase solvents might be selected from any vegetable oil, including but not limited to oleic, palmitic, steric or linoleic acid and glycerol, such as e.g. corn, olive, cottonseed, rapeseed, sesame, canola, safflower, sunflower, soybean, grapeseed, or peanut oil, preferably corn oil.

Carbon sources to be used for the present invention might be selected from linear alkanes, free fatty acids, ethanol, glucose and/or mixtures thereof.

Suitable host cells to be used for the present invention might be selected from host cells capable of retinoid production, particularly retinyl acetate-producing host cells, such as e.g. fungal host cells including oleaginous yeast cells, such as e.g. Rhodosporidium, Lipomyces or Yarrowia, preferably Yarrowia, more preferably Yarrowia lipolytica, preferably comprising one or more genetic modifications in the endogenous lipase activity, such as i.e. reduced or abolished activity of endogenous lipases involved in conversion of retinol into fatty acid retinyl esters (FAREs), one major undesired side-product in fermentative retinoid production, and/or expressing genes coding for heterologous enzymes EC class [EC 2.3.1.84] catalyzing the enzymatic conversion of retinol into retinyl acetate. Suitable strains expressing such acetyl transferases (ATFs) are described in e.g. WO2019058001 or WO2020141168.

Thus, in one embodiment the invention is related to fermentative production of retinoids including retinol and retinyl acetate, wherein the host cell grown on a suitable carbon source, including e.g. linear alkanes, free fatty acids, glucose, ethanol and/or mixtures thereof, is cultivated in the presence of a plant-based second phase, particularly vegetable oil, said host cell being modified in endogenous lipase activities, particularly, wherein the activity of one or more endogenous gene(s) encoding enzymes with activity equivalent to Yarrowia LIP2 and/or LIP3 and/or LIP4 and/or LIP8, is reduced or abolished, such as polypeptides with at least about 50%, such as 60, 70, 80, 90, 95, 98, or 100% identity to SEQ ID NO:1, 3, 5, 7, or combinations thereof, wherein SEQ ID NO:1 corresponds to LIP2 obtainable from Yarrowia lipolytica, SEQ ID NO:3 corresponds to LIP3 obtainable from Yarrowia lipolytica, SEQ ID NO:5 corresponds to LIP8 obtainable from Yarrowia lipolytica, SEQ ID NO:7 corresponds to LIP4 obtainable from Yarrowia lipolytica. Preferably, the process as defined herein comprising a vegetable oil as second phase as described herein is modified in the activity of a lipase corresponding to activity of Yarrowia LIP8, such as particularly with reduced or abolished activity, more particularly abolished activity, of a gene encoding a lipase with activity corresponding to LIP8 activity from Yarrowia lipolytica, more preferably wherein a polypeptide with at least about 50% identity to SEQ ID NO:5 is abolished.

As used herein, an enzyme, particularly a lipase as defined herein, having “reduced or abolished” activity means a decrease in its specific activity, i.e. reduced/abolished ability to catalyze formation of a product from a given substrate into glycerol and fatty acids during fermentation, including reduced or abolished activity of the respective (endogenous) gene encoding such lipases. A reduction by 100% is referred herein as abolishment of enzyme activity, achievable e.g. via deletion, insertions, frameshift mutations, missense mutations or premature stop-codons in the endogenous gene encoding said enzyme or blocking of the expression and/or activity of said endogenous gene(s) with known methods.

As used herein, “deletion” of a gene leading to abolishment of gene activity includes all mutations in the nucleic acid sequence that can result in an allele of diminished function, including, but not limited to deletions, insertions, frameshift mutations, missense mutations, and premature stop codons, wherein deleted means that the corresponding gene/protein activity, such as particularly endogenous lipase activity, cannot be detected (any more) in the host cell.

Genetic modifications as defined herein include, but are not limited to, e.g. gene replacement, gene amplification, gene disruption, transfection, transformation using plasmids, viruses, or other vectors. An example of such a genetic modification may for instance affect the interaction with DNA that is mediated by the N-terminal region of enzymes as defined herein or interaction with other effector molecules. In particular, modifications leading to reduced/abolished specific enzyme activity may be carried out in functional, such as functional for the catalytic activity, parts of the proteins. Furthermore, reduction/abolishment of enzyme specific activity might be achieved by contacting said enzymes with specific inhibitors or other substances that specifically interact with them. In order to identify such inhibitors, the respective enzymes, such as e.g. certain endogenous lipases as defined herein, may be expressed and tested for activity in the presence of compounds suspected to inhibit their activity.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

As used herein, an enzyme is “expressed and active in vivo” if mRNA encoding for the protein can be detected by Northern blotting and/or protein is detected by mass spectrometry. With regards to ATFs as defined herein it means ability of a host cell for acetylation of retinol into retinyl acetate.

In one aspect, the present invention is directed to a fermentation process using such lipase-modified host cell defined herein said host cell being cultivated in the presence of a plant-based second phase as defined herein, such as vegetable oil, particularly such as e.g. corn oil, said host cell being grown on a suitable carbon source as defined herein, wherein the production/accumulation of retinoids is increased by at least about 10% compared to a process comprising silicone oil as second phase.

The term “lipase” is used interchangeably herein with the term “enzyme having lipase activity”. It refers to enzymes involved in pre-digestion of triglyceride oils such as e.g. vegetable oil into glycerol and fatty acids that are normally expressed in oleaginous host cells. Suitable enzymes to be modified in a host cell as defined herein might be selected from endogenous enzymes belonging to EC class 3.1.1.-, including, but not limited to one or more enzyme(s) with activities corresponding to Yarrowia LIP2, LIP3, LIP4, or LIP8 activities.

As used herein, an enzyme having “activity corresponding to the respective LIP activity in Yarrowia” includes not only the genes originating from Yarrowia, e.g. Yarrowia lipolytica, such as e.g. Yarrowia LIP2, LIP3, LIP4, LIP8 or combinations thereof, but also includes enzymes having equivalent enzymatic activity but are originated from another source organism, particularly retinyl acetate-producing oleaginous host cell, wherein a modification of such equivalent endogenous genes would lead to an increase in retinol to retinyl acetate conversion as defined herein.

In one embodiment, the host cell to be used in the present invention might express further enzymes, such as heterologous acetylating enzymes (ATFs), particularly fungal ATF, comprising a highly conserved partial amino acid sequence of at least 7 amino acid residues selected from [NDEHCS]-H-x(3)-D-[GA] (motifs are in Prosite syntax, as defined in https://prosite.expasy.org/scanprosite/scanprosite_doc.html), wherein “x” denotes an arbitrary amino acid and with the central histidine being part of the enzyme's binding pocket, preferably wherein the 7 amino acid motif is selected from [NDE]-H-x(3)-D-[GA], more preferably selected from [ND]-H-x(3)-D-[GA], most preferably selected from N—H-x(3)-D-[GA] corresponding to position N218 to G224 in the polypeptide according to SEQ ID NO:1 in WO2020/141168. Examples of such enzymes might be particularly selected from L. mirantina, L. fermentati, S. bayanus, or W. anomalus, such as e.g. LmATF1 according to SEQ ID NO:1 in WO2020/141168, SbATF1, LffATF1, LfATF1, Wa1ATF1 or Wa3ATF1 as disclosed in WO2019/058001, more preferably said ATFs comprising one or more amino acid substitution(s) in a sequence with at least about 20%, such as e.g. 25, 30, 35, 40, 45, 50, 55, 60, 65, 70, 75, 80, 85, 90, 92, 95, 97, 98, 99% or up to 100% identity to SEQ ID NO:1 in WO2020141168, wherein the one or more amino acid substitution(s) are located at position(s) corresponding to amino acid residue(s) selected from the group consisting of position 68, 69, 72, 73, 171, 174, 176, 178, 291, 292, 294, 301, 307, 308, 296, 312, 320, 322, 334, 362, 405, 407, 409, 480, 483, 484, 490, 492, 520, 521, 522, 524, 525, 526 and combinations thereof and as particularly exemplified in Table 4 of WO2020141168, most preferably comprising one or more amino acid substitution(s) on positions corresponding to amino acid residue(s) 69, 407, 409, 480, 484, and combinations thereof in SEQ ID NO:1 in WO2020141168.

In one particular embodiment, the modified host cell to be used for the process according to the present invention comprises an amino acid substitution at a position corresponding to residue 69 in the ATF according to SEQ ID NO:1 in WO2020/141168 leading to asparagine, serine or alanine at said residue, such as e.g. via substitution of histidine by asparagine (H69N), serine (H69S) or alanine (H69A), with preference for H69A. Said modified enzyme might be originated from yeast, such as e.g. L. mirantina, L. fermentati, W. anomalus or S. bayanus, preferably from L. mirantina, optionally being combined with amino acid substitution at a position corresponding to residue 407 in the ATF according to SEQ ID NO:1 in WO2020141168 leading to isoleucine at said residue, such as e.g. via substitution of valine by isoleucine (V407I), optionally being combined with an amino acid substitution at a position corresponding to residue 409 in the ATF according to SEQ ID NO:1 in WO2020141168 leading to alanine at said residue, such as e.g. via substitution of glycine by alanine (G409A), optionally being combined with amino acid substitution at a position corresponding to residue 480 in the ATF according to SEQ ID NO:1 in WO2020141168 leading to glutamic acid, lysine, methionine, phenylalanine or glutamine at said residue, such as e.g. via substitution of serine by glutamic acid (S480E), lysine (S480L), methionine (S480M), phenylalanine (S480F) or glutamine (S480Q), optionally being combined with amino acid substitution at a position corresponding to residue 484 in the ATF according to SEQ ID NO:1 in WO2020141168 leading to leucine at said residue, such as e.g. via substitution of isoleucine by leucine (1484L). Said modified enzyme might be originated from yeast, such as e.g. L. mirantina, L. fermentati, W. anomalus or S. bayanus, preferably from L. mirantina. In a most preferred embodiment, the ATF to be used for the process according to the present invention is a modified ATF comprising amino acid substitutions S480Q_G409A_V407I_H69A_1484L and is obtainable from Lachancea mirantina.

The host cell as defined herein is cultivated together with the suitable carbon source, comprising e.g. linear alkanes, free fatty acids, glucose, ethanol and/or mixtures thereof, and in the presence of the plant-based second phase comprising e.g. vegetable oils, is cultured in an aqueous medium supplemented with appropriate nutrients under aerobic or anaerobic conditions and as known by the skilled person for the different host cells. The cultivation/growth of the host cell may be conducted in batch, fed-batch, semi-continuous or continuous mode. Depending on the host cell, preferably, production of retinoids such as e.g. vitamin A and precursors such as retinal, retinol, retinyl acetate can vary, as it is known to the skilled person. Cultivation and isolation of beta-carotene and retinoid-producing host cells selected from Yarrowia is described in e.g. WO2008042338.

Fermentation products including retinyl acetate that are sequestered outside the cell may be harvested from the cultivation at a suitable moment, e.g. when one or more of the nutrients are exhausted. Depending on the host cell, preferably, production of retinoids such as e.g. vitamin A, precursors and/or derivatives thereof such as retinal, retinol, retinyl acetate, particularly retinyl acetate, can vary, as it is known to the skilled person.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity. With regards to enzymes originated from plants, the skilled person knows plant-derived enzymes might contain a chloroplast targeting signal which is to be cleaved via specific enzymes, such as e.g. chloroplast processing enzymes (CPEs).

The enzymes as described herein to be expressed in a suitable host cell to be used in the present invention also encompass enzymes carrying (further) amino acid substitution(s) which do not alter enzyme activity, i.e. which show the same properties with respect to the enzymes defined herein. Such mutations are also called “silent mutations”. Examples of silent mutations included in the present invention are host-optimized sequences.

As used herein, “activity” of an enzyme, e.g. activity of lipases or ATFs as defined herein, is defined as “specific activity” i.e. its catalytic activity, i.e. its ability to catalyze formation of a product from a given substrate, such as e.g. the formation of retinyl fatty esters or retinyl acetate. An enzyme, e.g. a lipase, is active, if it performs its catalytic activity in vivo, i.e. within the host cell as defined herein according to the process as defined herein. The skilled person knows how to measure enzyme activity, in particular activity of lipases, ATFs or other enzymes as defined herein. Analytical methods to evaluate the capability of lipases as defined herein involved in formation of retinyl fatty esters are known in the art and include measurement via HPLC and the like. With regards to activity of LIP2, LIP3, LIP8, LIP4 as defined herein, the skilled person might measure the formation of retinyl fatty esters from conversion of retinol in comparison to the formation of retinyl acetate from conversion of retinol, both measured with a modified and the respective wild-type host cell. Analytical methods to evaluate the capability of a suitable ATF as defined herein for retinyl acetate production, i.e. acetylation of retinol, or enzymes with lipase activity as defined herein are known in the art, such as e.g. described in Example 4 of WO2014096992. In brief, titers of products such as retinyl acetate, retinol, trans-retinal, cis-retinal, beta-carotene and the like can be measured by HPLC.

In one embodiment, the two-phase culture system using a plant-based second phase solvent such as e.g. vegetable oil as second phase as described herein comprises cultivation of a suitable host cell, such as an oleaginous yeast, including Yarrowia, particularly wherein certain endogenous lipase activities have been reduced or abolished as defined herein, preferably wherein an enzyme with activity corresponding to Yarrowia LIP8 has been reduced or abolished, furthermore comprises one or more modifications in enzyme activity, such as e.g. expressing heterologous ATFs involved in conversion of retinol into retinyl acetate as described herein, such as in particular ATF originated from Lachancea or Saccharomyces.

Optionally, the host cell as defined herein, such as e.g. Yarrowia, particularly retinyl acetate-producing host cell, particularly Yarrowia, is expressing further enzymes used for biosynthesis of beta-carotene and/or additionally enzymes used for catalyzing conversion of beta-carotene into retinal and/or retinal into retinol. The skilled person knows which genes to be used/expressed for either biosynthesis of beta-carotene and/or bio-conversion of beta-carotene into retinol. Genes and methods to generate carotenoid-producing host cells are known in the art, see e.g. WO2006102342. Depending on the carotenoid to be produced, different genes might be involved.

Thus, in one embodiment the host cell used in the two-phase culture system as defined herein might express further enzymes used for biosynthesis of beta-carotene.

In a particular embodiment, the host cell as used in a process defined herein might be originated from Yarrowia lipolytica as disclosed in WO2019058001 or WO2019057999, thus further genetically modified, wherein the formation of retinyl acetate from beta-carotene is optimized via heterologous expression of beta-carotene oxidases (BCOs), retinol dehydrogenases (RDHs) and/or acetyl-transferases (ATFs). Particularly, a modified host cell to be used in a process as defined herein might be expressing a BCO originated from Drosophila melanogaster or Danio rerio, RDH originated from Fusarium, and fungal ATF, such as e.g. ATF originated from Lachancea or Saccharomyces, wherein the enzymes might be encoded by host-optimized nucleic acid sequences. To enhance the conversion of beta-carotene into retinal into retinol into retinyl acetate in a process as defined herein, said enzymes might comprise one or more mutations leading to improved enzyme activities, such as e.g. acetylation of retinol into retinyl acetate.

A host cell comprising the above-described modifications in endogenous lipase-activities and or ATF activities is also referred to as “modified host cell”. The terms “retinoid-producing host cell capable of retinoid or retinyl acetate formation” and “retinoid- or retinyl acetate-producing host cell” are used interchangeably herein.

As used herein, a “wild-type host cell” means the respective host cell which is wild-type, i.e. non-modified, with respect to the above-mentioned lipase activity and/or ATF modifications. Thus, in a wild-type host cell the corresponding endogenous enzymes as defined herein are (still) expressed and active in vivo and/or no heterologous enzymes are expressed.

In one embodiment the present invention is directed to a two phase culture system wherein a host cell as defined herein is cultivated in the presence of suitable carbon sources as defined herein and using a plant-based second phase, e.g. vegetable oil as second phase, i.e. wherein the retinyl acetate is accumulated in the second phase, particularly vegetable oil. Particularly, the second phase comprising the retinyl acetate can be used directly in a pharmaceutical, nutritional, cosmetic application or composition without any further purification or isolation steps.

Thus, the present invention is related to a lipophilic composition and the process for producing said lipophilic composition as defined herein, comprising vegetable oil and retinoids, particularly retinyl acetate, to be used in a pharmaceutical, feed, food, or cosmetic composition, with a percentage of retinyl acetate in the range of up to about 30%, such as e.g. from about 0.0001% to about 30%, particularly in the range of about 1, 5, 10, 15, 20, 25, 28, 30% based total amount in the composition.

The pharmaceutical, food, feed or cosmetic composition comprising vegetable oil and retinyl acetate might be used in a mixture with particularly further fat- or water-soluble vitamins, preferable fat-soluble vitamins, such as e.g. tocopherols, tocotrienols, carotenoids, calciferols, menadiones, ubiquinones thiamine, riboflavin, pyridoxine, cobalamin, ascorbate, niacin, pantothenic acid, biotin, and/or folate. Forms can be as oleaginous or aliphatic liquids, emulsions and composites in starch and other binders as known in the art.

Thus, according to one embodiment the present invention features a pharmaceutical, feed, food, cosmetic composition comprising vegetable oil, particularly corn oil, and retinyl acetate, particularly with a percentage in the range of about 0.001 to about 30% based on total retinoids in said composition, optionally comprising further ingredients selected from vitamins, such as further fat- and/or water-soluble vitamins, particularly fat-soluble vitamins, such as selected from one or more vitamins such as e.g. tocopherols, tocotrienols, carotenoids, calciferols, menadiones, ubiquinones, thiamine, riboflavin, pyridoxine, cobalamin, ascorbate, niacin, pantothenic acid, biotin, and/or folate, and a process for producing said pharmaceutical, feed, food, cosmetic compositions, said process comprising the steps of:
(1) cultivation of a suitable retinyl-acetate producing host cell, particularly fungal host cell, such as e.g. an oleaginous yeast, particularly Yarrowia, said host cell being cultivated in the presence of a plant-based second phase, e.g. vegetable oil, such as e.g. corn oil, as second phase solvent,
(2) isolation of the plant-based second phase, such as e.g. vegetable oil, particularly corn oil, comprising retinyl acetate, and
(3) formulation of said composition of step (2), and optionally,
(4) mixing with one or more fat- or water-soluble vitamin(s) selected from the group consisting of tocopherols, tocotrienols, carotenoids, calciferols, menadiones, ubiquinones, thiamine, riboflavin, pyridoxine, cobalamin, ascorbate, niacin, pantothenic acid, biotin, folate, and mixtures thereof.

As used herein, the term “formulation” means mixing with other vitamins, anti-oxidants and excipients and processing into a stable and bioavailable matrix for delivery in human and animal feed, pharma or cosmetic applications.

“Retinoids” or a “retinoid-mix” as used herein include vitamin A, precursors and/or intermediates of vitamin A such as beta-carotene cleavage products also known as apocarotenoids, including but not limited to retinal, retinoic acid, retinol, retinoic methoxide, retinyl acetate, retinyl fatty esters, 4-keto-retinoids, 3 hydroxy-retinoids or combinations thereof. Biosynthesis of retinoids is described in e.g. WO2008042338. A host cell capable of production of retinoids in e.g. a fermentation process is known as “retinoid-producing host cell”. The genes of the vitamin A pathway and methods to generate retinoid-producing host cells are known in the art (see e.g. WO2019058000), including but not limited to beta-carotene oxidases, retinol dehydrogenases and/or acetyl transferases. Suitable ATFs capable of acetylation of retinol into retinyl acetate are disclosed in e.g. WO2019058001 or WO2020141168. Suitable beta-carotene oxidases leading to high percentage of trans-retinal are described in e.g. WO2019057999. A “retinyl acetate-producing host cell” as used herein is expressing suitable ATFs catalyzing the conversion of retinol into retinyl acetate such that the host cell is capable of retinyl acetate accumulation.

The terms “triglycerides” and “triglyceride oils” are used interchangeably herein.

“FAREs” or “retinyl fatty esters” as used interchangeably herein includes long chain retinyl esters. These long chain retinyl esters define hydrocarbon esters that consists of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds.

Long chain retinyl esters include but are not limited to linoleic acid, oleic acid, or palmitic acid.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion. A preferred form of vitamin A is retinyl acetate, wherein the terms “retinyl acetate”, “retinol acetate” and “vitamin A acetate” might be used interchangeably (see https://www.cancer.gov/publications/dictionaries/cancer-drug/def/retinyl-acetate?redirect=true).

“Retinal” as used herein is known under IUPAC name (2E,4E,6E,8E)-3,7-Dimethyl-9-(2,6,6-trimethylcyclohexen-1-yl)nona-2,4,6,8-tetraenal. It includes both cis- and trans-isoforms, such as e.g. 11-cis retinal, 13-cis retinal, trans-retinal and all-trans retinal. For the purpose of the present invention, the formation of trans-retinal is preferred, which might be generated via the use of stereoselective beta-carotene oxidases, such as described in e.g. WO2019057999.

“Carotenoids” as used herein include long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342. Cells capable of carotenoid production via one or more enzymatic conversion steps leading to carotenoids, particularly to beta-carotene, i.e. wherein the respective polypeptides involved in production of carotenoids are expressed and active in vivo are referred to herein as carotenoid-producing host cells. The genes and methods to generate carotenoid-producing cells are known in the art, see e.g. WO2006102342. Depending on the carotenoid to be produced, different genes might be involved.

“Conversion” according to the present invention is defined as specific enzymatic activity, i.e. catalytic activity of enzymes described herein, including but not limited to the enzymatic activity of lipases, in particular enzymes belonging to the EC class 3.1.1.-involved in conversion of retinol into retinyl fatty esters, beta-carotene oxidases (BCOs), retinol dehydrogenases (RDHs), acetyl transferases (ATFs).

With regards to the present invention, it is understood that organisms, such as e.g. microorganisms, fungi, algae, or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

The following examples are illustrative only and are not intended to limit the scope of the invention in any way. The contents of all references, patent applications, patents, and published patent applications, cited throughout this application are hereby incorporated by reference, in particular WO2020141168, WO2019058001, WO2008042338, WO2014096992, WO2006102342, WO2019057999, WO2016172282.

### EXAMPLES

### Example 1: General Methods and Strains

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998). All genetic manipulations exemplified were performed in Yarrowia lipolytica.

Shake plate assay. Typically, 200 μl of 0.075% Yeast extract, 0.25% peptone (0.25×YP) is inoculated with 10 μl of freshly grown Yarrowia and overlaid with 200 μl of Drakeol 5 (Penreco, Karns City, PA, USA) mineral oil, silicone oil, or corn oil with either 2% oleic acid or 2% glucose as a carbon source. Clonal isolates of transformants were grown in 24 well plates (Multitron, 30° C., 800 RPM) in YPD media with one of the overlays indicated earlier for 4 days. The overlay fraction was removed from the shake plate wells and analyzed by HPLC on a normal phase column, with a photo-diode array detector.

DNA transformation. Strains are transformed by overnight growth on YPD plate media 50 μl of cells is scraped from a plate and transformed by incubation in 500 μl with 1 μg transforming DNA, typically linear DNA for integrative transformation, 40% PEG 3550MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells are out grown on YPD liquid media for 4 hours at 30° C. before plating on the selective media. Nourseothricin (Nat) selection was performed on YPD media containing 100 μg/mL nourseothricin and hygromycin (Hyg) selection was performed on YPD containing 100 μg/mL hygromycin. URA3 marker recycling was performed using 5-fluoroorotic acid (FOA). Episomal hygromycin resistance marker (Hyg) plasmids were cured by passage on non-selective media, with identification of Hyg-sensitive colonies by replica plating colonies from non-selective media to hygromycin containing media (100 μg/mL).

DNA molecular biology. Plasmid MB9523 containing expression systems for DrBCO, LmATF-S480Q_G409A_V407I_H69A_1484L, and FfRDH (SEQ ID NO:10) was synthesized at Genscript (Piscataway, NJ, USA). Plasmid MB9523 contains the ‘URA3’ for marker selection in Yarrowia lipolytica transformations. For clean gene insertion by random nonhomologous end joining of the gene and marker a Sfil plasmid fragment of interest from MB9523 was purified by gel electrophoresis and Qiagen gel purification column. Clones were verified by sequencing. Typically, genes are synthesized by a synthetic biology at GenScript (Piscataway, NJ). Plasmid MB8388-LIP8 (SEQ ID NO:11), containing a Cas9, and guide RNA expression systems to target LIP8, was synthesized at Genscript (Piscataway, NJ, USA).

Plasmid list. Plasmid, strains, nucleotide and amino acid sequences that were used are listed in Table 1, 2 and the sequence listing. In general, all non-modified sequences referred to herein are the same as the accession sequence in the database for reference strain CLIB122 (Dujon B, et al, Nature. 2004 Jul. 1; 430(6995):35-44).

TABLE 1
list of plasmids used for construction of the strains for
overexpression or deletion of the respective genes
indicated as ″insert″. ″LmATF1-mut″ refers to
Lachancea mirantina (LmATF1; SEQ ID NO:13
in WO2019058001) carrying aa substitutions
S480Q_G409A_V407I_H69A_I484L. ″DrBCO″
refers to BCO originated from Danio rerio
(see SEQ ID NO:18 in WO2020141168); ″FfRDH″
refers to RDH originated from Fusarium (see SEQ ID NO:
22 in WO2020141168). For more explanation, see text.

| | | | SEQ |
|---|---|---|---|
| Plasmid | Insert | Marker | ID NO: |
| MB7452 | Cas9 | Nat | 9 |
| MB9523 | DrBCO; LmATF1-mut; FfRDH | URA3 | 10 |
| MB8388-LIP8 | Cas9; LIP8 targeting guide RNAs | Hyg | 11 |

TABLE 2
list of Yarrowia lipolytica strains used. Construction of ML17544 is
described in Table 2 of WO2020141168. For more details, see text.

| Strain | Description |
|---|---|
| ML18812 | ML17544 transformed with MB9523 |
| ML18812-lip8 | ML18812 with MB7542 and MB8388-LIP8, lip8 deletion |

Retinoid quantification. Analysis of retinoids were carried out with a C4 reverse phase retinoid method (see below) and C18 as described elsewhere (WO2020141168). The addition of all added intermediates gives the total amount of retinoids.

C4 reverse phase chromatography. For exact determination of discrete retinoids the long run reverse phase system was used. We separated analytes at 230 nm and 325 nm through the Agilent 1290 instrument with YMC Pro C4, 150×3.0 mm 3 μm column (YMC America, Allentown PA) stationary phase, and a 5 μl injection loop volume and column and sample tray controlled at 23° C. with gradients described in Table 4B. Analytes were detected at 230 nm and 325 nm and the peaks identity verified with LCMS. The analytes separated as discrete peaks that were assigned according to Table 4A.

TABLE 4A
list of analytes using C4-reverse phase method. The addition
of all added intermediates gives the total amount retinoids.
″RT″ means retention time. For more details, see text.

| Intermediates | RT [min] | λ max [nm] |
|---|---|---|
| trans-retinol | 20.21 | 325 |
| cis-retinol | 20.32 | 325 |
| dihydro-retinol | 20.75 | 290 |
| trans-retinal | 20.89 | 380 |
| cis-retinal | 21.02 | 380 |
| trans-retinyl-acetate | 22.15 | 325 |
| cis-retinyl-acetate | 22.35 | 325 |
| dihydro-retinyl acetate | 22.60 | 290 |
| retinyl esters | 26-30 | 325 |

TABLE 4B
UPLC Method Gradient with
solvent A: acetonitrile;
solvent B: water;
solvent C: water/acetonitrile/
methanesulfonic acid 1000:25:1.
For more details, see text.

| Time | % | % | % | Flow |
|---|---|---|---|---|
| [min] | A | B | C | [ml/min] |
| 0 | 5 | 85 | 10 | 0.5 |
| 20 | 98 | 0 | 2 | 0.5 |
| 35 | 98 | 0 | 2 | 0.5 |
| 35.1 | 5 | 85 | 10 | 0.5 |
| 40 | 5 | 85 | 10 | 0.5 |

Method Calibration. Method is calibrated using high purity retinyl acetate received from DSM Nutritional Products, Kaiseraugst, CH. Retinols and retinal are quantitated against retinyl acetate. Dilutions described in Table 4C are prepared as follows. 40 mg of retinyl acetate is weighed into a 100 mL volumetric flask, and dissolved in ethanol, yielding a 400 μg/mL solution. This solution is sonicated as required to ensure dissolution. 5 mL of this 400 μg/mL solution is diluted into 50 mL (1/10 dilution, final concentration 40 μg/mL), 5 mL into 100 mL (1/20 dilution, final concentration 20 μg/mL), 5 mL of 40 μg/mL into 50 mL (1/10 dilution, final concentration 4 μg/mL), 5 mL of 20 μg/mL into 50 mL (1/10 dilution, 2 μg/mL), using 50/50 methanol/methyl tert-butyl ether (MTBE) as the diluent. All dilutions are done in volumetric flasks. Purity of retinyl acetate is determined by further diluting the 400 μg/mL stock solution 100-fold (using a 2 mL volumetric pipet and a 200 mL volumetric flask) in ethanol. Absorbance of this solution at 325 nm using ethanol is taken as the blank, with adjustment of the initial concentration using the equation (Abs*dilution (100)*molecular weight (328.5)/51180=concentration in mg/mL). Because of quick out-maximization of UV absorbance of retinyl acetate, lower concentrations are better.

TABLE 4C
preparation of calibration standards.
For more explanation, see text.

| Stock [RA], | Final |
|---|---|
| dilution | concentration |
| 20 μg/mL, 1/10 | 2 μg/ml |
| 40 μg/mL, 1/10 | 4 μg/mL |
| 400 μg/mL, 1/20 | 20 μg/mL |
| 400 μg/ml, 1/10 | 40 μg/mL |

Sample preparation. Top second phase layer samples from each strain were diluted at a 25-fold dilution or higher into tetrahydrofuran (THF). Fermentation whole broth was prepared using a 2 mL Precellys (Bertin Corp, Rockville, MD) tube, add 25 μl of well mixed broth and 975 μl of THF. Precellys 3×15×7500 rpm for two cycles with a freeze at −80° C. for 10 minutes between cycles. Cell debris was spun down via centrifugation for 1 minute at 13000 rpm. These samples were diluted 10-fold in THF.

Fermentation conditions. Fermentations used a silicone oil or corn oil overlay and stirred tank that was glucose fed in a bench top reactor with 0.5 L to 5 L total volume (see WO2016172282, Ex. 5 and 6). Generally, the same results were observed with a fed batch stirred tank reactor with an increased productivity demonstrating the utility of the system for the production of retinoids. Preferably, fermentations were batched with 6% glucose and 20% silicone oil or 20% corn oil was added after dissolved oxygen dropped below about 20% and feed was resumed to achieve 20% dissolved oxygen throughout the feeding program. Fermenters were harvested and compared at 138 h.

### Example 2: Deletion of LIP8 in Yarrowia lipolytica to Enable Use of Corn Oil as a Second Phase

Lipase gene LIP8 was disabled in strain ML18812 using modern CRISPR Cas9 methods, to generate strain ML18812-LIP8. Briefly, strain ML18812 was transformed with MB7452 (SEQ ID NO:9), which contains an expression module for Cas9 and the nourseothricin selection marker. Nourseothricin resistant transformants (selected on YPD with 200 μg/mL nourseothricin) were subsequently transformed with plasmid MB8388-LIP8 (SEQ ID NO:11), which contains expression sequences for Cas9, and guide RNAs with seed sequences targeting LIP8, and the hygromycin resistance marker. Transformants (selected on YPD with 200 μg/mL hygromycin) were screened for mutation by Sanger sequencing with primers flanking the targeted region. Strain ML18812-LIP8 was found to have inactivating mutations in LIP8.

Strains ML18812, which is wild-type strain, i.e. wherein the endogenous lipase genes are still active, and strain ML18812-LIP8, which carries the LIP8 deletion, were growing in a fermentor as described in Example 1, with glucose as carbon source and either corn oil or silicone as second phase. Second phase was measured for the presence of retinoids, the results are shown in Table 5.

TABLE 5
retinoid production in strain ML18812-LIP8
(deletion of LIP8) compared to wild-type strain
ML18812 (″LIP+″) as control together with
silicone oil or corn oil as second phase.
The number for ML18812 with silicone
oil is set to 100%, the other numbers are
calculated in relation thereto.
For more explanation, see text.

| | Relevant | Total retinoids | |
|---|---|---|---|
| | lipase | (% of silicone oil) | |
| Strain | genotype | Silicone oil | Corn oil |
| ML18812 | LIP+ | 100 | 6.3 |
| ML18812-LIP8 | deletion lip8 | 95 | 110 |

While both strains produced comparable levels of retinoids in silicone oil, strains with LIP8 deletion/inactivation had significantly higher retinoids in corn oil second phase than strains with wild-type LIP8.

In addition, the volume of second phases from each of the four conditions was measured. Results are shown in Table 6 below.

TABLE 6
impact of LIP8 deletion on different second-phase
solvents. For more explanation, see Table 5 or text.

| | | % second phase remaining | |
|---|---|---|---|
| | Relevant | at end of fermentation | |
| | lipase | compared to beginning | |
| Strain | genotype | Silicone oil | Corn oil |
| ML18812 | LIP+ | >95% | <5% |
| ML18812-LIP8 | deletion lip8 | >95% | >95% |

While the second phase amounts were unchanged between WT and LIP8-deletion strains for silicone oil, strains with wild-type LIP8 had a marked reduction in the amount of corn oil second phase remaining in the fermentor. Strains in which LIP8 was inactivated did not have any significant reduction in the amount of corn oil second phase over the course of the fermentation.

TABLE 7
sequences. SEQ ID NOs: 2, 4, 6, 8 refer to
the polynucleotides expressing LIP2, LIP3,
LIP4, LIP8, respectively, according to
SEQ ID NOs: 1, 3, 5, 7. for more
explanation, see text.

| SEQ | |
|---|---|
| ID | |
| NO: | sequence 5′ to 3′ |
| 1 | MKLSTILFTA CATLAAALPS PITPSEAAVL QKRVYTSTET SHIDQESYNE |
| | FEKYARLANI GYCVGPGTKI FKPFNCGLQC AHFPNVELIE EFHDPRLIED |
| | VSGYLAVDHA SKQIYLVIRG THSLEDVITD IRIMQAPLTN VTGHSLGGAA |
| | ATCDDCLVHN GFIQSYNNTY NQIGPKLDSV IEQYPDYQIA FDLAANISST |
| | ALLFGINLKV NGHDPLVVTL GQPIVGNAGF ANWVDKLFFG QENPDVSKVS |
| | KDRKLYRITH RGDIVPQVPF WDGYQHCSGE VFIDWPLIHP PLSNVVMCQG |
| | QSNKQCSAGN TLLQQVNVIG NHLQYFVTEG VCGI |
| 2 | atgaagcttt ccaccatcct cttcacagcc tgcgctaccc tggctgccgc |
| | cctcccttcc cccatcactc cttctgaggc cgcagttctc cagaagcgag |
| | tgtacacctc taccgagacc tctcacattg accaggagtc ctacaacttc |
| | tttgagaagt acgcccgact cgcaaacatt ggatattgtg ttggtcccgg |
| | cactaagatc ttcaagccct tcaactgtgg cctgcaatgt gcccacttcc |
| | ccaacgttga gctcatcgag gagttccacg acccccgtct catctttgat |
| | gtttctggtt acctcgctgt tgatcatgcc tccaagcaga tctaccttgt |
| | tattcgagga acccactctc tggaggacgt cataaccgac atccgaatca |
| | tgcaggctcc tctgacgaac tttgatcttg ctgctaacat ctcttctact |
| | gctacttgtg atgactgtct tgtccacaat ggcttcatcc agtcctacaa |
| | caacacctac aatcagatcg gccccaagct cgactctgtg attgagcagt |
| | atcccgacta ccagattgct gtcaccggtc actctctcgg aggagctgca |
| | gcccttctgt tcggaatcaa cctcaaggtt aacggccacg atcccctcgt |
| | tgttactctt ggtcagccca ttgtcggtaa cgctggcttt gctaactggg |
| | tcgataaact cttctttggc caggagaacc ccgatgtctc caaggtgtcc |
| | aaagaccgaa agctctaccg aatcacccac cgaggagata tcgtccctca |
| | agtgcccttc tgggacggtt accagcactg ctctggtgag gtctttattg |
| | actggcccct gatccaccct cctctctcca acgttgtcat gtgccagggc |
| | cagagcaata aacagtgctc tgccggtaac actctgctcc agcaggtcaa |
| | tgtgattgga aaccatctgc agtacttcgt caccgagggt gtctgtggta |
| | tctaa |
| 3 | MPLELPSLNA SIVGNTVQNG AVEQFLNIRY ADIPGKFEKP VLKNDWNGAE |
| | IDATKVGPVC PQPRTPFNFF SVPDDLWEKV NVDTYQDGLL CDNLIVTRPK |
| | GVSANARLPT VVWIHGGSNI EGSIYNLIYE PQFLVAESVR VGKPIVHVCI |
| | EYRLGLAGFL TKNGKGNWGT WDQYTGCQWV NRHIQDFGGD PLNVTLTGES |
| | AGSVAVHNML IKDSMNGRKL FRNAVMMSGT LETITPQPPK WHARLEEKVA |
| | KVTGKEVADL ASLSDKELLD AQIKLNVAVC MTCDDGDFFE PGWKQHLTPD |
| | WLDKLIISDC KDEGMLYFLP VNAQDDEELL AKVAKSPVGK EISELYGIKE |
| | GGDIKSACLD LKTDATFNYF NHLLEKKMEE ARNNGSTSRV YRLAVDEPNP |
| | HNPDQRAHHA VDVLYMENST KFNEHGDKLS RLFQSHELRL AYGLEPWDHR |
| | NFGVYRNGGY QQLPLSELNK VRPVERYEAL SKMDFGQVGR LSNALSRL |
| 4 | atgcctctcg aactcccctc gctcaacgcc tcgattgtcg gcaacaccgt |
| | tcagaacggc gctgttgagc agtttctcaa catccgatac gccgacattc |
| | ctggcaagtt tgagaagccc gtgctcaaga acgattggaa cggcgcggag |
| | atcgacgcca ccaaggtcgg tcccgtgtgc ccccaacccc gcaccccatt |
| | caacttcttc tccgtgccag acgacctctg ggagaaagtc aatgtggaca |
| | cgtaccagga cggtctgctg tgcgacaacc tgattgtgac gcgaccgaag |
| | ggcgtgtctg ccaacgcccg gctgcccact gttgtgtgga tccacggcgg |
| | ctccaatatt gagggcagta tctacaacct catctatgag ccccagttcc |
| | tggtggcaga gtcggtgcga gtaggcaagc cgattgtgca cgtgtgtatc |
| | gagtaccgac tgggtctcgc gggcttcctc accaagaacg gcaagggcaa |
| | ctggggcacg tgggatcagt acacgggctg ccagtgggtc aaccgccaca |
| | ttcaggactt tggaggcgat cctttgaacg tgacattgac cggtgagtct |
| | gccggctctg tagcagtcca taacatgctc atcaaggact ccatgaacgg |
| | ccgaaagttg ttccgaaatg ccgtcatgat gagtggcact ctcgagacca |
| | tcactcctca gcctcccaag tggcatgctc gtttggagga gaaggtggcc |
| | aaggtcactg gcaaggaagt ggccgacctt gcttctctgt ccgataagga |
| | gctgctcgac gcccagatca agctcaatgt ggctgtgtgc atgacttgcg |
| | acgacggcga ctttttcgag cccggatgga agcagcatct gactcctgac |
| | tggctcgaca agctcatcat ctccgattgc aaggacgagg gcatgctgta |
| | tttcctgcca gtcaacgcgc aggacgacga ggagctgttg gcaaaggtgg |
| | ccaagtcgcc cgtgggtaag gagatttccg agctttacgg catcaaggag |
| | ggtggcgata tcaagtctgc gtgtctcgat ctcaagactg acgccacctt |
| | caattacttt aaccatctgc tgttcaagaa gatggaggag gcccgaaaca |
| | acggctccac ttctcgagtt taccgtctgg ccgtcgatga gcccaacccc |
| | cacaaccccg accagcgggc ccaccacgcc gtcgacgtgc tgtacatgtt |
| | caactcgacc aagttcaacg agcacggcga caagctgtct cggctgttcc |
| | agagccactt tttgcggctg gcgtatggcc tggagccctg ggaccatcga |
| | aactttggag tgtacagaaa cggcggctac cagcagctgc cgctgagtga |
| | gttgaacaag gtccgacccg tcgagcggta cgaggcgctg tccaagatgg |
| | actttggcca ggttgggcgt ttgtccaatg cgctttcgcg cctatga |
| 5 | MVSLSARIKD FFSVLLLGAA TITPSTQTAG VSQGFYDFAR DFAHLSNIAY |
| | CVNAPITPLN PDFTCGNSCK HFPEIELVKT FGGNFFKTSI TGYLAVDHVK |
| | KEKYVVERGT FSLADAITDM QFQLSPELVD VPALNTFSAN DTTAEAQTHC |
| | EGCKIHDGES KAFTETWGNI GEDLQKHLDA NPDYQLYVTG HSLGAAMALL |
| | GATSIKLKGY DPILINYGQP RVGNKPFAEF INKLWFGEGN GLEITPERKL |
| | YRMTHWNDIF VGLPNWEGYT HSNGEVYINN RFINPPLKDV ISCAGGENSK |
| | CYRSSESLLS QINLLQNHLA YIDYIGYCAL NIGRRELADQ EHYTGPYYYG |
| | HRSEEDFKKL GLELSTPQVE N |
| 6 | atggtatccc tctctgctcg aatcaaagac tttttttcgg tcctcctcct |
| | cggagctgca accatcactc cctccacaca gaccgcaggc gtgtctcaag |
| | ggttctatga ttttgctcgg gactttgccc atctgtccaa cattgcctac |
| | tgtgtcaatg ctcccatcac tccactgaac ccggacttca cctgtggcaa |
| | ctcgtgcaag cactttccgg aaattgagct tgtgaagaca tttggaggca |
| | acttcttcaa gacctccatt acgggctacc tggctgtcga tcatgtcaag |
| | aaggagaagt acgttgtctt ccgaggaacc ttttcgctgg cagacgcgat |
| | cacggacatg cagttccagc tgtctccttt cctggtcgat gtgcctgccc |
| | tgaacacttt ctcagctaat gacaccaccg cagaggccca gacgcactgt |
| | gagggctgca aaattcacga cggcttctcc aaggccttta ccgagacctg |
| | gggtaacatt ggtgaggatc tgcagaaaca cctggacgct aacccggact |
| | accagctgta cgtgactggc cattctctgg gagctgctat ggcccttctt |
| | ggagctactt ccatcaagct caagggctac gatcccattc tcatcaacta |
| | cggacagccc cgagtcggaa acaagccctt cgctgagttc attaacaagt |
| | tgtggtttgg agaaggcaac ggtctggaaa tcacccccga gagaaagctg |
| | taccgaatga cccactggaa cgacatcttt gttggcctgc ccaactggga |
| | gggatacacc cactctaacg gtgaagtata catcaacaac cggttcatca |
| | accctcctct caaggatgtc atctcttgtg ctggaggcga aaactcgaag |
| | tgctaccgat cctcgttcag cctgctgtcc cagatcaatc tgctccaaaa |
| | ccacctggct tacattgatt acattggata ctgcgctctg aacattggtc |
| | gacgagagct tgccgatcag gaacattaca ctggtcctta ttactatggt |
| | catcgatctg aggaggactt taagaagttg ggcttggagc tatccacccc |
| | acaagttgag aactga |
| 7 | MAGFNFTFGQ VISYLASMLY GQVDATSSST RIQATQDLYD FTAKFSRLSN |
| | IAYCINAPFT PLRTDFTCGE SCRYFPDLQL DSVFGGNFSS ASTTGYIAYD |
| | HKKKEKYIVF RGTFSIPDII TDIQFQTAPW LTSLPTHLIP TKEDFEHKQA |
| | ILKHYAAENK GLSNLEERQD VVHEDPSLVP KKMDKCENCQ IHDGFAKGFN |
| | ETIEHAGPQI EKFLGNNTDY KMFVVGHSLG AAQAQLFATQ FKLLGFDPYM |
| | INFGQPRIGN PEFAAYINQL WFNDTGLVVN DARRFYRVTH WNDIVVGVPD |
| | WLNYTHSIGE VFIDEESVYP KLDKVVVCEG GENPLCHRGT FNLWSRINEL |
| | QNHLAYIFYI GLCAFNIGRR DVLNMPQYQG NFSYQHNIDP NYNYDTKVPT |
| | RISKSN |
| 8 | atggcagggt tcaacttcac tttcgggcag gtgatttcgt acctggcttc |
| | catgctctat ggacaggtgg atgccacttc atcctccact cgaatccagg |
| | ccacccagga tctgtacgac ttcacagcca agttttcgcg actctcaaac |
| | atcgcgtact gcatcaatgc ccccttcacg cctctcagaa cggacttcac |
| | ctgcggagaa agctgtcggt acttccccga cctccagctg gactcagtgt |
| | ttggtggtaa cttctcctca gcctccacta ccggctacat tgcatacgac |
| | cacaagaaga aggaaaagta cattgtgttt cgaggaactt tcagtatccc |
| | tgatatcatc acagacattc aatttcagac tgccccttgg ttgacctctc |
| | tgcccacgca tctgatccct accaaggagg actttgaaca caagcaggcc |
| | atcctgaagc actacgctgc cgaaaacaag ggtctcagca acctggaaga |
| | gcgacaggat gttgtgcatg aagaccctag cctggttccc aagaaaatgg |
| | acaagtgcga gaactgccag atccatgacg gattcgccaa gggcttcaac |
| | gagactatcg agcatgccgg accccagatt gaaaagttcc tgggcaataa |
| | caccgactac aagatgtttg ttgttggcca ctctctagga gctgctcagg |
| | cccagctgtt tgctacacag ttcaaactgc tgggatttga cccttacatg |
| | atcaactttg gacagcctcg acttggaaac cctgagttcg ccgcctacat |
| | caaccagctg tggttcaacg atactggtct ggttgtcaat gatgcccgac |
| | gattctaccg agtgactcac tggaacgata tcgtcgtggg agtgcccgac |
| | tggctcaatt acacccactc tatcggagag gtgttcatag acgaggagag |
| | cgtttacccc aagctggaca aggtggtggt gtgcgaggga ggagagaacc |
| | ccctgtgcca ccgaggaact ttcaacctgt ggtcacgaat caacttcctg |
| | cagaaccatt tggcttatat cttctacatt ggtctgtgtg ctttcaacat |
| | tggccgaaga gacgtgctca acatgccaca ataccagggc aacttctcgt |
| | accagcacaa catcgacccc aactacaatt acgataccaa ggttcccacc |
| | cggatcagta aatcaaacta a |
| 9 | cgcgtggatc gccggtgcgt tgacgttggt gacctccagc cagaggtgcc |
| | cggcgccccg ctcgccggcg aactccgtcg cgagccccat caacgcgcgc |
| | ccgaccccgt gcccccggtg ctccggggcg acctcgatgt cctcgacggt |
| | cagccggcgg ttccacgccg agtacgagat gaccacgaag cccgccaggt |
| | cgccgtcgtc cccgtacgcg acgaacgtcc gggagtccgg gtcgccgtcc |
| | tccccgtcgt ccgattcgtc gtccgattcg tcgtcgggga acaccttggt |
| | caggggcggg tccaccggca cctcccgcag ggtgaagccg tccccggtgg |
| | cggtgacgcg gaagacggtg tcggtggtga aggacccatc cagtgcctcg |
| | atggcctcgg cgtcccccgg gacactggtg cggtaccggt aagccgtgtc |
| | gtcaagagtg gtcatttttg tgtctaggtg tttgtgtttg gactgcgatc |
| | agtgaagaaa agaagaggaa aaattgtgca agaaattttg ctttcaagac |
| | ttggctgatg cagcagggta actctgggac acagacctat gtttgtggtt |
| | aaactcaatg cacgtggtac gtgcgtggag cgcttaccca tccaagggtg |
| | tggacatgga accgacggtc cgtggagttg tgtaatgtca ttttggcgac |
| | tcttgaagca aggctataaa aaaattgtgt ggcttgagtc ttatcgagct |
| | cggtcactac aagagttaat cttcctgtct caggcagaca ggtcaggcag |
| | ggttactttt gggtgtgctg taactcactg tatggccgtt agtgcgcata |
| | gacgttgtac atactggacc gaattgtagc gtgctcaata gggccaataa |
| | agctattgta gggatccgaa ttttcagaac ctaatttatc tgttacccgg |
| | cctgtggctc gcacagctta aaaatggtca aactttcccc ttcttgtctt |
| | tttttcctca cattcatcag gttcttgtct tgatctttca agtgagtatt |
| | aattaccgac cttggttctt cattgggaga gcattggaag ccgtggtgca |
| | gcaaccacaa aacggttctt ccccttcgat accttcttgc ctgcctttca |
| | atacaagtcg gctcgattag cggtggtcgc ccccgccagc ggagaacatg |
| | gaactaaccc agaatgagag ctaagtggag aaagaagaga gtcagacgac |
| | tcaagcgaaa gcgccgcaag gtccgagctc gatccaaata agcggttttt |
| | aacggagatt taacactaaa tcgaagaact tttcccgttt catttgcgaa |
| | tgagctcgtt aacaaaatcc cccagttttt ttatccagct gtaaggattg |
| | acattagtaa tgaattattg tttggtatat ttaaatctgt agttcctttc |
| | tgtccgtgtc ggcaactgtc gtactcgtga tttacttgta ttgacgaata |
| | cttactgtag cgcactctgc tgctactggt cgtaaggatg tgctatttcg |
| | gtgtatggtg ggttttttgg gggtcggaac cgaagactgt tacacgggca |
| | cggctcgttg tgtacacgca cagagctctt gcgagtcatg ttgtagctag |
| | ctcgtcgtgt tcaggaactg ttcgatggtt cggagagagt cgccgcccag |
| | aacatacgcg caccgatgtc agcagacagc cttattacaa gtatattcaa |
| | gcaagtatat ccgtagggtg cgggtgattt ggatctaagg ttcgtactca |
| | acactcacga gcagcttgcc tatgttacat ccttttatca gacataacat |
| | aattggagtt tacttacaca cggggtgtac ctgtatgagc accacctaca |
| | attgtagcac tggtacttgt acaaagaatt tattcgtacg aatcacaggg |
| | acggccgccc tcaccgaacc agcgaatacc tcagcggtcc cctgcagtga |
| | ctcaacaaag cgatatgaac atcttgcgat ggtatcctgc tgatagtttt |
| | tactgtacaa acacctgtgt agctccttct agcattttta agttattcac |
| | acctcaaggg gagggataaa ttaaataaat tccaaaagcg aagatcgaga |
| | aactaaatta aaattccaaa aacgaagttg gaacacaacc ccccgaaaaa |
| | aaacaacaaa caaaaaaccc aacaaaataa acaaaaacaa aataaatata |
| | taactaccag tatctgacta aaagttcaaa tactcgtact tacaacaaat |
| | agaaatgagc cggccaaaat tctgcagaaa aaaatttcaa acaagtactg |
| | gtataattaa attaaaaaac acatcaaagt atcataacgt tagttatttt |
| | attttattta ataaaagaaa acaacaagat gggctcaaaa ctttcaactt |
| | atacgataca taccaaataa caatttagta tttatctaag tgcttttcgt |
| | agataatgga atacaaatgg atatccagag tatacacatg gatagtatac |
| | actgacacga caattctgta tctctttatg ttaactactg tgaggcatta |
| | aatagagctt gatatataaa atgttacatt tcacagtctg aacttttgca |
| | gattacctaa tttggtaaga tattaattat gaactgaaag ttgatggcat |
| | ccctaaattt gatgaaagat gaaattgtaa atgaggtggt aaaagagcta |
| | cagtcgtttt gttttgagat accatcatct ctaacgaaat atctattaaa |
| | aatctcagtg tgatcatgag tcattgccat cctggaaaat gtcatcatgg |
| | ctgatatttc taactgttta cttgagataa atatatattt acaagaactt |
| | cccttgaaat taatttagat ataaaatgtt tgcgggcaag ttactacgag |
| | gaataaatta tatctagagg ttccgcttcc tcgctcactg actcgctgcg |
| | ctcggtcgtt cggctgcggc gagcggtatc agctcactca aaggcggtaa |
| | tacggttatc cacagaatca ggggataacg caggaaagaa catgtgagca |
| | aaaggccagc aaaaggccag gaaccgtaaa aaggccgcgt tgctggcgtt |
| | tttccatagg ctccgccccc ctgacgagca tcacaaaaat cgacgctcaa |
| | gtcagaggtg gcgaaacccg acaggactat aaagatacca ggcgtttccc |
| | cctggaagct ccctcgtgcg ctctcctgtt ccgaccctgc cgcttaccgg |
| | atacctgtcc gcctttctcc cttcgggaag cgtggcgctt tctcatagct |
| | cacgctgtag gtatctcagt tcggtgtagg tcgttcgctc caagctgggc |
| | tgtgtgcacg aaccccccgt tcagcccgac cgctgcgcct tatccggtaa |
| | ctatcgtctt gagtccaacc cggtaagaca cgacttatcg ccactggcag |
| | cagccactgg taacaggatt agcagagcga ggtatgtagg cggtgctaca |
| | gagttcttga agtggtggcc taactacggc tacactagaa ggacagtatt |
| | tggtatctgc gctctgctga agccagttac cttcggaaaa agagttggta |
| | gctcttgatc cggcaaacaa accaccgctg gtagcggtgg tttttttgtt |
| | tgcaagcagc agattacgcg cagaaaaaaa ggatctcaag aagatccttt |
| | gatcttttct acggggtctg acgctcagtg gaacgaaaac tcacgttaag |
| | ggattttggt catgagatta tcaaaaagga tcttcaccta gatcctttta |
| | aattaaaaat gaagttttaa atcaatctaa agtatatatg agtaaacttg |
| | gtctgacagt taccaatgct taatcagtga ggcacctatc tcagcgatct |
| | gtctatttcg ttcatccata gttgcctgac tccccgtcgt gtagataact |
| | acgatacggg agggcttacc atctggcccc agtgctgcaa tgataccgcg |
| | agacccacgc tcaccggctc cagatttatc agcaataaac cagccagccg |
| | gaagggccga gcgcagaagt ggtcctgcaa ctttatccgc ctccatccag |
| | tctattaatt gttgccggga agctagagta agtagttcgc cagttaatag |
| | tttgcgcaac gttgttgcca ttgctgcagg catcgtggtg tcacgctcgt |
| | cgtttggtat ggcttcattc agctccggtt cccaacgatc aaggcgagtt |
| | acatgatccc ccatgttgtg caaaaaagcg gttagctcct tcggtcctcc |
| | gatcgttgtc agaagtaagt tggccgcagt gttatcactc atggttatgg |
| | cagcactgca taattctctt actgtcatgc catccgtaag atgcttttct |
| | gtgactggtg agtactcaac caagtcattc tgagaatagt gtatgcggcg |
| | accgagttgc tcttgcccgg cgtcaacacg ggataatacc gcgccacata |
| | gcagaacttt aaaagtgctc atcattggaa aacgttcttc ggggcgaaaa |
| | ctctcaagga tcttaccgct gttgagatcc agttcgatgt aacccactcg |
| | tgcacccaac tgatcttcag catcttttac tttcaccagc gtttctgggt |
| | gagcaaaaac aggaaggcaa aatgccgcaa aaaagggaat aagggcgaca |
| | cggaaatgtt gaatactcat actcttcctt tttcaatatt attgaagcat |
| | ttatcagggt tattgtctca tgagcggata catatttgaa tgtatttaga |
| | aaaataaaca aataggggtt ccgcgcacat ttccccgaaa agtgccacct |
| | gacgtctaag aaaccattat tatcatgaca ttaacctata aaaataggcg |
| | tatcacgagg ccctttcgtc tggcctagga agcgacttcc aatcgctttg |
| | catatccagt accacaccca caggcgtttg tgctactcta ctgatagcaa |
| | tagatgcgtc ataattggtt ggcccgctga gcctccacag gatactattg |
| | cacataccct ggtcatgtgc agatcagctc atttgtggag actctggagt |
| | aacttagacg acgcctggtt caattgccgc aatgtgcgcc cacgcagata |
| | atgtattgag gggtggagcg cctcttgggg acttgctgta cttgtacggg |
| | atattaaacg cactcagcaa gaccatgacg taaaacacac ctactgtacg |
| | atacgtactg taggtattgt actcgtaccc ggtactacaa atagtacgat |
| | actatacgga gtgtatttgt accttgatat acgactggcg gagtgaagag |
| | aaggagttga acaagaccag atggggatat cagccccagt gctttgtatt |
| | acaagtacga gtacttaata gatactgtaa ggctattgat acggatggca |
| | gtaagtcatt gagtaagcaa ttgtggccca gcatctcccc tacgtacttg |
| | taccataccc catggagaca ccaatggtct ttcacgcaca ctgtcgtgtg |
| | ctgtatcgca gaatcgggtg tccaaccaaa tgccgttacc cccacgtcac |
| | agccgataga cagatacacc atcaatacca gcaggttgta tcatgcggtt |
| | ggctgaaggt aagctgattg gtctaaaaac tgtagctgtc ctaattcaac |
| | gagcgctatt tggggccaac cacctcggcc aagcggcctt taatctgcgt |
| | gccccagagg cgtctaatga ggctctggcc gccactgtag gagtgtttct |
| | ctgtgcgcac acgcagtttt gagtttgggc gactttccct ttttcccaat |
| | tgcgtacaca cacagctccg agctaagcgc tgtccttgaa ccttctccct |
| | cttttccctc tttttctctt ccccttcccc tcctccacat taaggccaaa |
| | tcctgaattg caccaactag tacaacgaca acaatggaca agaagtactc |
| | catcggtttg gacattggta ctaactctgt cggctgggcc gtcatcaccg |
| | acgagtacaa ggttccctcc aagaagttca aggtccttgg caacaccgac |
| | cgacactcta tcaagaagaa cctgatcggt gctctgctgt tcgactctgg |
| | cgagactgcc gaggccaccc gactgaagcg aaccgctcga cgccgataca |
| | cccgacgaaa gaaccgaatc tgttacctcc aggagatctt cagcaacgag |
| | atggctaagg tcgacgactc cttcttccac cgactcgagg agtctttcct |
| | ggtcgaagag gataagaagc acgagcgaca ccccatcttc ggcaacattg |
| | ttgatgaggt tgcctaccat gagaagtacc ccaccatcta ccacctccga |
| | aagaagctcg tcgactccac tgacaaggct gacctccgac tcatctacct |
| | tgctctcgcc cacatgatca agttccgagg tcacttcctc attgagggtg |
| | atctcaaccc cgacaactcc gacgttgaca agctgttcat ccagctcgtc |
| | cagacctaca accagctctt tgaggagaac cctatcaacg cttctggtgt |
| | tgacgccaag gccattctct ccgcccgact ctctaagtcc cgacgactcg |
| | agaacctcat tgcccagctg cccggcgaga agaagaacgg cctcttcggt |
| | aacctgattg ctctctctct tggtctgacc cccaacttca agtccaactt |
| | tgacctcgcc gaggacgcca agctccagct gtccaaggac acctacgatg |
| | acgatctgga caacctcctg gcccagatcg gtgaccagta cgccgatctc |
| | ttccttgccg ccaagaacct ctccgacgcc atcctgctct ccgacatcct |
| | ccgagtcaac accgagatta ccaaggctcc tctgtctgcc tctatgatca |
| | agcgatacga cgagcaccac caggatctca ctcttctcaa ggctctcgtc |
| | cgacagcagc tccccgagaa gtacaaggag attttctttg accagtccaa |
| | gaacggttac gctggctaca ttgacggtgg tgcttcccag gaagagtttt |
| | acaagttcat caagcctatt ctggagaaga tggacggtac cgaggagctg |
| | ctcgtcaagc tcaaccgaga ggacctcctt cgaaagcagc gaaccttcga |
| | taacggctcc atcccccacc agatccacct gggtgagctc cacgccattc |
| | tccgaagaca agaggacttc taccccttcc taaaggataa ccgagagaag |
| | atcgagaaga ttctcacctt ccgaatcccc tactacgtcg gtcccctcgc |
| | tcgaggtaac tcccgatttg cttggatgac ccgaaagtcc gaggagacta |
| | tcaccccctg gaactttgaa gaggtagtcg acaagggtgc ctccgcccag |
| | tctttcattg agcggatgac caacttcgat aagaacctcc ccaacgagaa |
| | ggtccttccc aagcactctc tcctctacga gtacttcacc gtctacaacg |
| | agctgaccaa ggtcaagtac gttaccgagg gcatgcgaaa gcccgctttc |
| | ctctctggtg agcagaagaa ggccattgtc gacctcctgt tcaagactaa |
| | ccgaaaagtc accgtcaagc agctcaagga agactacttc aagaagattg |
| | agtgcttcga ctccgtcgag atttccggtg tcgaggaccg attcaacgcc |
| | tccctcggca cctaccacga tcttctgaag atcatcaagg acaaggactt |
| | tcttgataac gaggagaacg aggacattct cgaggacatc gtcctcaccc |
| | tcaccctttt cgaggatcga gagatgatcg aggagcgact caagacctac |
| | gcccatctct tcgacgacaa ggtcatgaag caactcaagc gacgacgata |
| | cactggctgg ggccgacttt cccgaaagct catcaacggc atccgagaca |
| | agcagtctgg caagaccatc ctggacttcc tgaagtccga cggtttcgcc |
| | aaccgaaact tcatgcagct catccacgac gactctctta ccttcaaaga |
| | ggatatccag aaggcccagg tttctggcca gggcgactcc ctccacgagc |
| | acattgccaa cctcgccgga tcccccgcca tcaaaaaggg tatcctccag |
| | accgtcaagg ttgtcgacga actcgtgaag gtcatgggcc gacacaagcc |
| | cgagaacatc gttatcgaga tggcccgaga gaaccagacc acccagaagg |
| | gtcagaagaa ctcccgagag cgaatgaagc gaatcgaaga gggtatcaag |
| | gagctcggtt cccagattct caaggagcac cccgtcgaga acacccagct |
| | ccagaacgag aaactctacc tgtactacct ccagaatggc cgagacatgt |
| | acgttgacca ggagctcgac atcaaccgac tctccgacta cgacgtcgac |
| | cacattgttc ctcagtcctt cctcaaggac gactccatcg acaacaaggt |
| | tctgacccga tctgacaaga accgaggtaa gtccgacaac gttccctccg |
| | aagaggtcgt taagaagatg aagaactact ggcgacagct tctcaacgcc |
| | aaactgatca cccagcgaaa gtttgacaac ctcaccaagg ccgagcgagg |
| | tggtctgtcc gagctggaca aggccggctt cattaagcga cagctggtcg |
| | agactcgaca gatcaccaag cacgtcgccc agatcctcga ctcccgaatg |
| | aacaccaagt acgacgagaa cgacaagctc atccgggagg tcaaggtcat |
| | caccctgaag tctaagcttg tctccgactt ccgaaaggac ttccagttct |
| | acaaggtccg agagatcaac aactaccacc acgcccacga cgcctacctc |
| | aacgccgttg ttggtaccgc cctcatcaag aagtatccca agctcgagtc |
| | cgagttcgtt tacggcgact acaaggttta cgatgtccga aagatgattg |
| | ccaagtccga gcaggagatc ggtaaggcca ccgccaagta ctttttctac |
| | tccaacatca tgaatttctt caagaccgag atcactctcg ccaacggtga |
| | gattcgaaag cgacccctga ttgagactaa tggtgagact ggtgagatcg |
| | tctgggataa gggccgagac ttcgccaccg tccgaaaggt cctgtccatg |
| | ccccaggtca acattgtcaa gaagaccgag gtccagaccg gtggcttctc |
| | caaggagtcc attctcccca agcgaaactc cgacaaactc atcgcccgta |
| | agaaggactg ggatccgaag aagtacggtg gtttcgattc tcccaccgtt |
| | gcctactccg tcctcgttgt tgctaaagtc gagaagggta agtctaagaa |
| | actcaagtcc gtgaaggagc tactcggtat caccatcatg gagcgatctt |
| | cttttgagaa gaaccccatt gacttcctcg aggccaaggg ttacaaagag |
| | gtcaagaagg acctgattat caagctgccc aagtactccc tctttgagct |
| | cgagaacggc cgaaagcgaa tgctggcttc cgctggtgag ctgcagaagg |
| | gcaacgagct cgctctgccc tccaagtacg tcaacttcct ctacctggcc |
| | tcccactacg agaagctcaa gggctccccc gaggacaacg agcagaagca |
| | gctgttcgtt gagcagcaca agcactacct cgacgagatc atcgagcaga |
| | tctccgagtt ctccaagcga gtcatcctcg ctgacgccaa ccttgataag |
| | gttctctctg cttacaacaa gcaccgggac aagcccatcc gagagcaggc |
| | cgagaatatc atccacctct tcactctcac caacctcggc gctcctgctg |
| | ccttcaagta cttcgacacc accattgacc gaaagaggta cacctccacc |
| | aaggaagtcc tcgacgccac cctgatccac cagtccatca ccggcctcta |
| | cgaaacccga atcgacctct cccagctcgg cggtgactct cgagccgacc |
| | ccaagaagaa gcgaaaagtc taaatatccg aagatcaaga gcgaagcaag |
| | ttgtaagtcc aggacatgtt tcccgcccac gcgagtgatt tataacacct |
| | ctcttttttg acacccgctc gccttgaaat tcatgtcaca taaattatag |
| | tcaacgacgt ttgaataact tgtcttgtag ttcgatgatg atcatatgat |
| | tacattaata gtaattactg tatttgatat atatactaat tacaatagta |
| | catattagaa catacaatag ttagtgccgt gaagtggctt aaaataccgc |
| | gagtcgatta cgtaatatta ttacctcttg cccatcgaac gtacaagtac |
| | tcctctgttc tctccttcct ttgctttgtg cacgaagaac tgcggtcagg |
| | tgacacaact ttttccatct cagggtgtgt cgcgtgtgct tcatccaaac |
| | tttagttggg gttcgggttc gcgcgagatg atcacgtgcc ctgatttggt |
| | gtcgtccccc gtcgcgctgc gcacgtgatt tatttatttc cggtggctgc |
| | tgtctacgcg gggccttctc tgcccttctg tttcaacctt cgggcggttc |
| | tcgtaaccag cagtagcaat ccatttcgaa actcaaagag ctaaaaacgt |
| | taaacctcag cagtcgctcg acgaatgggc tgcggttggg aagcccacga |
| | ggcctatagc cagagcctcg agttgacagg agcccagacg ccttttccaa |
| | cggcaacttt tatataaaat ggcaatgtat tcatgcaatt gcggccgtgt |
| | caggttggag acactggacc acactctcca ttgcttcctg aggagatgga |
| | tcattgctag tgcatctacg cgcagcaatc ccgcaagctc gacaaccgta |
| | gatgggcttt ggtgggccaa tcaattacgc aacccgcacg ttaaattgta |
| | tgaggaagga aggccacggt acaaagtggg tggtcttcac ccagtggttg |
| | ttggtggcgt catgcagacc atggccgcca gtgtgctgga attgaatatt |
| | taccgttcgt ataatgtatg ctatacgaag ttataccggt ctcgtagtgt |
| | tcacgttcag ttcacggtga gcttaaaact atcttcaaga agagatttga |
| | gacctgattt atacttgcag caatgtttac ttcttatcgc gatacacgaa |
| | tgtgatacgg atcaaagtaa gcaggactac gataagataa cgaatgcggt |
| | gcagtccatg tcgattaggt atagatacat ttattttgtg ttatgttaca |
| | ttttgggggg atactgtcct acttgtagta cctacttgta gtggcgcgtt |
| | aggggcaggg catgctcatg tagagcgcct gccgctcgcc gtccgaggcg |
| | gtgccgtcgt acagggcggt gtccaggccg cagagggtga accccatccg |
| | ccggta |
| 10 | GCGGAGCCTAGGCCGGCCAGCGTGGCGCGTGGCCATATTGGCCAGCAGCTTACTACAGCTCAGCC |
| | AAAGTGGATCATGATGGACGGAAATATCGGACTGGAGGCCAAAAAGGAGGTGCTCAAATACGCAC |
| | GGGACAAGTCTGCACAGGTGGCATTCGAACCCACGTCTGTCCCCAAAGCCGCTGCTCTTTCCGAG |
| | CTCAATCTGCCCGTTTACCCCAACAACTCCATTGCCTTAGCCACCCCCAACACTGCAGAGCTCGA |
| | GGCCATGTTCGAGGCATTCCACGAGAAGGGCCGATTTGACGTTGACGACTGGTTTCCAGTCATAG |
| | ACGGTCTTGCTCTCGGAGCTGACTTTAGAAACGGAGCCACCATGTTGTCGCACCAACATCGTGGA |
| | CTCAAAGCTATTCTCGAACAAGGAACTCTGGCCCAGGCAATCCACATGCTACCATACATCCCAAC |
| | TTTGATCATCAAGGGCGGAGCCAACGGCGTCGTCGTCTTCCAACTCATTGATGATATCGAGTCTG |
| | CCATTCATTCACAGCGTTCTGCTTCCAATAAAACGCCTGGCCTGTTCCAGAAGGGCAACGCTGCG |
| | AGTGGAAACACCAAGGTCGGCGTCTACATGCAGTACTTTGAGCCCGAAGAAGTGGGCAGTCAGTC |
| | GATTGTAAGCGTGACTGGAGCTGGAGACACTCTGTTTGGAACCCTGGCCATGGAGATTGTCAAGG |
| | ACGAGTCCTGGTTGAACGATATGGGAGACAAGAAGAGTGCAGTTGTTTCTCGAGCCATGAACAAT |
| | GCTGTGAAGACTATTCAGAGTAAGGACGCTGTCTGCAAGAGCATCCTTTAAGTGATTTGCCATGC |
| | TTTCTCTTCTTCCACGATGTAAATACTTATTTTACACACTACTGTGCAGTAGCAAATACAGAACA |
| | AGAGTTGTCGCCTATTGACAGTACAGTACGAGTAGTGTATGTACAGTAGTTATACAATATCTATG |
| | TGAAATTCGTCGGCAGCTTTCGGCTGATGAACTACGAGTTCTTCGTAATAAATCATCAACGTAAT |
| | AAGCTTGGTACCAGAGACGGGTTGGCGGCGTATTTGTGTCCCAAAAAACAGCCCCAATTGCCCCA |
| | ATTGACCCCAAATTGACCCAGTAGCGGGCCCAACCCCGGCGAGAGCCCCCTTCACCCCACATATC |
| | AAACCTCCCCCGGTTCCCACACTTGCCGTTAAGGGCGTAGGGTACTGCAGTCTGGAATCTACGCT |
| | TGTTCAGACTTTGTACTAGTTTCTTTGTCTGGCCATCCGGGTAACCCATGCCGGACGCAAAATAG |
| | ACTACTGAAAATTTTTTTGCTTTGTGGTTGGGACTTTAGCCAAGGGTATAAAAGACCACCGTCCC |
| | CGAATTACCTTTCCTCTTCTTTTCTCTCTCTCCTTGTCAACTCACACCCGAAATCGTTAAGCATT |
| | TCCTTCTGAGTATAAGAATCATTCGCTAGCCACAAAAATGGGTGATCTCGATGCCCGAGGAACCT |
| | CTGCTCACCCCGAGCTCTCTGAGCGACCTTCTATTATGCCTTCTATGTCTGATATTCAGGACCCT |
| | TCTGGTGATGACAAGGCTACTCCCCGAGGATCTGCTGCTGGTCTGCCCCAGCTTGAGCTTGCTGG |
| | ACACGCCCGACGACTTGGCCACCTTGAGAACTTCTTTGCTGTCCAGGCTCGACAGCAGATTTACT |
| | CTTCTTTTGCTGTTTTTTGTGAGTTTGACACTGCTTGTTCTCTCGCTCAGCTTGCTTCTGCTGTG |
| | CGAAACGTTTGTCTTTCTAACCCCCTTCTCCTTCACACTGTTGAGCCTAAGCACCCTGACATCGC |
| | TGGATTCTACCACTCTGACGAGTACCTTTCCCGACCTTGGCCTCAGCACGATTACATGCGAGTTC |
| | TTCGAGAGGTTCACGTCGCTGACGTTGTTATGAACGGACAGAAGGAGCACGCTCACGTTGTTCGA |
| | GATGCTGTTGACGTTTTTCAGGCTCACGGAAACCAGGTTACTTCTGAGCTCCTTGAGCTTATGAC |
| | TCAGATTGAGATTCCTCACGCTTCTCAGACTCGACCCTCTTGGCGACTTCTCTGTTTTCCCCACG |
| | GAGAGGCTAACCGATGGCGAACCTTTGCTTTTGTTTCTAACCACTGTTCTTCTGATGGTCTTTCT |
| | GGTCTTAACTTCTTTCGAGATCTCCAGAAGGAGCTTGCTCACGGCCCCACCTCTGGTGCTCCTGG |
| | TGCCCCCGGAGCTTCCGGAGTTATTTTCGATTACGCTCAGGACGCTGCTACCCTGCCCAAGCTGC |
| | CCCCTCCCATTGATCAGAAGCTCGATTACCGACCTTCTAAGAAGGCTCTTCTCGGCCTTCTCGCT |
| | GGCAAGTTCGTTCGAGAGAAGCTCGGTTACGTTTCTGCTGCTCCTCCCACTACCCCTACCTCTGA |
| | CCTTGCTCACCCTGAGGGTCACCAGTACTACTGTTACCTTGTTAACGTTCCCACTTCTTCTGTTG |
| | CCCACATTAAGACTCAGGTGCGAGAGAACGTTCCTCACAAGTGTACTCTCACTCCCTTTCTCCAG |
| | GCTTGTTGGCTTGTTTCTCTGTTCAAGTACGGTCGAGTTTTTTCTGGTTCTTGGCTTGAGCGATA |
| | CACCGATGTTCTTGTTGCTATGAACACTCGACAGCTTCTCCCCGAGGACCTTGAGCTTCAGCGAC |
| | AGTACCGATACGGTTCTAACATTGGAGCTGTTCGATACAACTACCCTATTGCTCCCCTTGACGTT |
| | CGAGATAACGATCAGAAGTTCTGGTCCCTTGTTGAGTCTTACCGACTTGCCCTTTCTGATGCCCG |
| | AGATAAGAACGATTACCTTTACGCTCTTGGTGCTCTTATGCTCCCTGAGATTTACGAGAAGAAGA |
| | ACGTTGATGCTGTTGTTAACGATACCATTCTTAACCAGCGACGACAGGGAACCCTTCTTTCTAAC |
| | GTTGGTTACGTTCGAGATGAGCAGCCCACTGCTTTTGCTATTAAGAACCACGTTTTTTCTCAGGG |
| | AGTTGGAGCTAACCGAAACGCTTTTGTTCTTAACATTTGTGCTACCGATCAGGGTGGTCTTAACA |
| | TCGCTATTTCTATTGCTAAGGGAACCCTTGCTTCTCGACAGGAGGGACAGGAGCTTTGTGATATT |
| | TTTAAGTCTACTCTCCTTCGATTTTAAACGCGTCTATCCGAAGATCAAGAGCGAAGCAAGTTGTA |
| | AGTCCAGGACATGTTTCCCGCCCACGCGAGTGATTTATAACACCTCTCTTTTTTGACACCCGCTC |
| | GCCTTGAAATTCATGTCACATAAATTATAGTCAACGACGTTTGAATAACTTGTCTTGTAGTTCGA |
| | TGATGATCATATGATTACATTAATAGTAATTACTGTATCTGTACCTGCTGTGGACCACGCACGGC |
| | GGAACGTACCGTACAAATATTTTCTTGCTCACATGACTCTCTCTCGGCCGCGCACGCCGGTGGCA |
| | AATTGCTCTTGCATTGGCTCTGTCTCTAGACGTCCAAACCGTCCAAAGTGGCAGGGTGACGTGAT |
| | GCGACGCACGAAGGAGATGGCCCGGTGGCGAGGAACCGGACACGGCGAGCCGGCGGGAAAAAAGG |
| | CGGAAAACGAAAAGCGAAGGGCACAATCTGACGGTGCGGCTGCCACCAACCCAAGGAGGCTATTT |
| | TGGGTCGCTTTCCATTTCACATTCGCCCTCAATGGCCACTTTGCGGTGGTGAACATGGTTTCTGA |
| | AACAACCCCCCAGAATTAGAGTATATTGATGTGTTTAAGATTGGGTTGCTATTTGGCCATTGTGG |
| | GGGAGGGTAGCGACGTGGAGGACATTCCAGGGCGAATTGAGCCTAGAAAGTGGTACCATTCCAAC |
| | CGTCTCAGTCGTCCGAATTGATCGCTATAACTATCACCTCTCTCACATGTCTACTTCCCCAACCA |
| | ACATCCCCAACCTCCCCCACACTAAAGTTCACGCCAATAATGTAGGCACTCTTTCTGGGTGTGGG |
| | ACAGCAGAGCAATACGGAGGGGAGATTACACAACGAGCCACAATTGGGGAGATGGTAGCCATCTC |
| | ACTCGACCCGTCGACTTTTGGCAACGCTCAATTACCCACCAAATTTGGGCTGGAGTTGAGGGGAC |
| | CGTGTTCCAGCGCTGTAGGACCAGCAACACACACGGTATCAACAGCAACCAACGCCCCCGCTAAT |
| | GCACCCAGTACTGCGCAGGTGTGGGCCAGGTGCGTTCCAGATGCGAGTTGGCGAACCCTAAGCCG |
| | ACAGTGTACTTTTTGGGACGGGCAGTAGCAATCGTGGGCGTAGACCCCGGTGTATATAAAGGGGT |
| | GGAGAGGACGGATTATTAGCACCAACACACACACTTATACTACATGCTAGCCACAAAAATGCTCT |
| | CTTTCTTCTGGCGAAACGGTATCGAGACTCCCGAGCCCCTCAAGGCTGACGTTTCCGGCTCTATC |
| | CCTCCCTGGCTTCAGGGAACCCTTCTCCGAAACGGTCCTGGTCTGTTCTCCGTTGGCAACACTTC |
| | CTACAAGCACTGGTTCGATGGTATGGCTCTCATTCACTCCTTCACCTTTAAGGATGGTGAGGTTT |
| | TTTACCGATCTAAGTACCTGAAGTCTGAGACTTACAAGAAGAACATCGCTGCCGACCGAATCGTT |
| | GTGTCTGAGTTCGGAACCATGGTGTACCCCGATCCCTGCAAGAACATTTTCTCCCGAGCCTTCTC |
| | TTACATGATGAACGCCATTCCTGACTTTACCGATAACAACCTCATTAACATCATTAAGTACGGTG |
| | AGGATTACTACGCCTCCTCTGAGGTCAACTACATCAACCAGATTGACCCCCTGACCCTTGAGACT |
| | CTCGGACGAACTAACTACCGAAACCACATTGCCATCAACCTTGCCACTGCTCACCCTCACTACGA |
| | CGAGGAGGGTAACACTTACAACATGGGCACTGCTATTATGAACCTCGGTCGACCCAAGTACGTGA |
| | TTTTCAAGGTGCCCGCCAACACCTCTGATAAGGAGAACAAGAAGCCTGCCCTCTCTGAGGTGGAG |
| | CAGGTTTGCTCCATTCCCATCCGACCCTCCCTTTACCCTTCTTACTTCCACTCTTTTGGCATGAC |
| | TGAGAACTACATCATCTTCGTTGAGCAGGCCTTCAAGCTGGACATCGTCAAGCTGGCTACTGCTT |
| | ACTTCCGAGATATTAACTGGGGATCTTGCCTTAAGTTCGACCAGGATGACATTAACGTGTTCCAC |
| | CTGGTCAACAAGAAGACTGGTAAGGCTGTGTCCGTGAAGTACTACACTGACCCCTTTGTTACCTT |
| | CCACCACATCAACGCTTACGAGGACGATGGCCACGTCGTCTTCGATCTCATTACTTACAAGGACT |
| | CTAAGCTGTACGATATGTTCTACATTCAGAACATGAAGCAGGACGTCAAGCGATTTATTGAGACT |
| | AACAAGGACTTCGCTCAGCCCGTGTGCCAGCGATTTGTCCTTCCCGTCAACGTTGATAAGGAGAC |
| | TCCTCAGGACATCAACCTTGTCAAGCTGCAGGACACCACTGCCACTGCTGTCCTGAAGGAGGACG |
| | GCTCTGTCTACTGCACCCCTGACATCATTTTTAAGGGTCTTGAGCTCCCTGCTATCAACTACAAG |
| | TTTAACTCTAAGAAGAACCGATACTTCTACGGCACCCGAGTGGAGTGGTCCCCTTACCCTAACAA |
| | GGTCGCTAAGGTGGACGTTGTTACTCGAACCCACAAGATTTGGACTGAGGAGGAGTGTTACCCTT |
| | CTGAGCCTGTCTTTATTGCCTCCCCTGACGCCGTTGATGAGGATGACGGTGTGATTCTTTCTTCT |
| | GTGGTTTCTTTCAACCCCCAGCGACCCCCTTTCCTGGTTGTCCTCGATGCTAAGTCCTTCAAGGA |
| | GATTGCTCGAGCTACCATCGATGCCTCTATTCACATGGACCTTCACGGCCTTTTCATCCACGACA |
| | AGTCTACCTAAGTTTTTTGATCAATGATCCAATGGCTTTCACATACCCCCCCACGCCTATAATTA |
| | AAACACAGAGAAATATAATCTAACTTAATAAATATTACGGAGAATCTTTCGAGTGTTCAGCAGAA |
| | ATATAGCCATTGTAACAAAAGCCGGCTATCGACCGCTTTATCGAAGAATATTTCCCGCCCCCCAG |
| | TGGCCAAACGATATCGGTCAGAAGGGGCAGCTCTAAACGAAGAACTGCGGTCAGGTGACACAACT |
| | TTTTCCATCTCAGGGTGTGTCGCGTGTGCTTCATCCAAACTTTAGTTGGGGTTCGGGTTCGCGCG |
| | AGATGATCACGTGCCCTGATTTGGTGTCGTCCCCCGTCGCGCTGCGCACGTGATTTATTTATTTC |
| | CGGTGGCTGCTGTCTACGCGGGGCCTTCTCTGCCCTTCTGTTTCAACCTTCGGGCGGTTCTCGTA |
| | ACCAGCAGTAGCAATCCATTTCGAAACTCAAAGAGCTAAAAACGTTAAACCTCAGCAGTCGCTCG |
| | ACGAATGGGCTGCGGTTGGGAAGCCCACGAGGCCTATAGCCAGAGCCTCGAGTTGACAGGAGCCC |
| | AGACGCCTTTTCCAACGGCAACTTTTATATAAAATGGCAATGTATTCATGCAATTGCGGCCGTGT |
| | CAGGTTGGAGACACTGGACCACACTCTCCATTGCTTCCTGAGGAGATGGATCATTGCTAGTGCAT |
| | CTACGCGCAGCAATCCCGCAAGCTCGACAACCGTAGATGGGCTTTGGTGGGCCAATCAATTACGC |
| | AACCCGCACGTTAAATTGTATGAGGAAGGAAGGCCACGGTACAAAGTGGGTGGTCTTCACCCAGT |
| | GGTTGTTGGTGGCGTCATGCAGACCATGCATTGGGGATAGCACAGGGTTGGGGTGTCTTGTGGAC |
| | TCAATGGGTGAAAGGAGATGGAAAAGGGCGGTGAAAAGTGGTAGAATCGAAATCCCTGACGTCAA |
| | TTTATAAAGTAAAATGCGTTTCTGCCATTTTGCTCCCCTCCTTCTTTCGCAATCGCCTCCCCAAA |
| | AGTTGTCGTGGCAGTACACATGCTTGCATACAATGAAGCTAATCCGGCTTGCTCAGTAGTTGCTA |
| | TATCCAGGCATGGTGTGAAACCCCTCAAAGTATATATAGGAGCGGTGAGCCCCAGTCTGGGGTCT |
| | TTTCTCTCCATCTCAAAACTACTTTCTCACATGCTAGCCACAAAAATGACCACTAAGTACACTTC |
| | CGTTCACGAGTCTCCCAACGGCCCTGGTGACGCTCGACCCACCGCTTCCCAGATTATCGACGATT |
| | ACAACCTTGAGGGAGAGCTTTCTGGCAAGACTGTTCTCGTCACCGGCTGTTCCTCTGGTATTGGT |
| | GTTGAGACTGCCCGAGCTATTTACCGAACTGGTGCCACCCTTTACCTCACTGCCCGAGATGTCGA |
| | TAAGGCCAAGACCGTTCTTCCCGACCTTGTTGACACTTCCCGAGTCCACTTTCTCCACCTTGACC |
| | TTAACTCTCTGGAGTCTGTTCGAGGTTTTGCTGAGAACTTCAAGTCTAAGTCCACTCAGCTTCAC |
| | ATTCTCATCGAGAACGCTGGCGTGATGGCCTGTCCCGAGGGCCGAACCGTCGATGGTTTTGAGAC |
| | TCAGTTTGGTATCAACCACCTTGCTCACTTTCTCCTCTTTTACCTCCTCAAGGATACCCTTCTCA |
| | ACTCTTCTACCCCCGCTTTCAACTCCCGAGTTGTCATCCTCTCTTCTTGTGCTCACCAGGCTGGT |
| | TCCGTTCACCTTAACAACCTGTCTCTTGAGGGTGGATACGAGCCTTGGAAGTCTTACGGCCAGTC |
| | CAAGACTGCCAACCTTTGGACTGCCCGAGAGATCGAGAAGCGATTTGGTGCTTCCGGTATCCACT |
| | CTTGGGCTGTTCACCCCGGTTCCATCGCTACTGAGCTTCAGCGACACGTTTCCGACGAGCTTAAG |
| | CAGAAGTGGGCTGACGATAAGGAGGGTGCCAAGCTGTGGAAGTCCACCGAGCAGGGTGCCGCCAC |
| | CACTGTCCTTGCTGCTGTTTCCCCTGAGCTTGAGGGTAAGGGCGGTCTTTACCTTGAGGATACCC |
| | AGGTTGCCAAGCCCCCTGCCCGAGGAATGTTTGGTGTTGCTGACTGGGCTTACGATGAGGATGGC |
| | CCCTCTAAGCTCTGGGCCAAGTCTCTTGAGCTCCTTAAGCTCCAGTAAAGGTTAGACTATGGATA |
| | TGTAATTTAACTGTGTATATAGAGAGCGTGCAAGTATGGAGCGCTTGTTCAGCTTGTATGATGGT |
| | CAGACGACCTGTCTGATCGAGTATGTATGATACTGCACAACCTGTGTATCCGCATGATCTGTCCA |
| | ATGGGGCATGTTGTTGTGTTTCTCGATACGGAGATGCTGGGTACAAGTAGCTAATACGATTGAAC |
| | TACTTATACTTATATGAGGCTTGAAGAAAGCTGACTTGTGTATGACTTATTCTCAACTACATCCC |
| | CAGTCACAATACCACCACTGCACTACCACTACACACTAGTGGTGTGTTCTGTGGAGCATTCTCAC |
| | TTTTGGTAAACGACATTGCTTCAAGTGCAGCGGAATCAAAAAGTATAAAGTGGGCAGCGAGTATA |
| | CCTGTACAGACTGTAGGCGATAACTCAATCCAATTACCCCCCACAACATGACTGGCCAAACTGAT |
| | CTCAAGACTTTATTGAAATCAGCAACACCGATTCTCAATGAAGGCACATACTTCTTCTGCAACAT |
| | TCACTTGACGCCTAAAGTTGGTGAGAAATGGACCGACAAGACATATTCTGCTATCCACGGACTGT |
| | TGCCTGTGTCGGTGGCTACAATACGTGAGTCAGAAGGGCTGACGGTGGTGGTTCCCAAGGAAAAG |
| | GTCGACGAGTATCTGTCTGACTCGTCATTGCCGCCTTTGGAGTACGACTCCAACTATGAGTGTGC |
| | TTGGATCACTTTGACGATACATTCTTCGTTGGAGGCTGTGGGTCTGACAGCTGCGTTTTCGGCGC |
| | GGTTGGCCGACAACAATATCAGCTGCAACGTCATTGCTGGCTTTCATCATGATCACATTTTTGTC |
| | GGCAAAGGCGACGCCCAGAGAGCCATTGACGTTCTTTCTAATTTGGACCGATAGCCGTATAGTCC |
| | AGTCTATCTATAAGTTCAACTAACTCGTAACTATTACCATAACATATACTTCACTGCCCCAGATA |
| | AGGTTCCGATAAAAAGTTCTGCAGACTAAATTTATTTCAGTCTCCTCTTCACCACCAAAATGCCC |
| | TCCTACGAAGCTCGAGCTAACGTCCACAAGTCCGCCTTTGCCGCTCGAGTGCTCAAGCTCGTGGC |
| | AGCCAAGAAAACCAACCTGTGTGCTTCTCTGGATGTTACCACCACCAAGGAGCTCATTGAGCTTG |
| | CCGATAAGGTCGGACCTTATGTGTGCATGATCAAGACCCATATCGACATCATTGACGACTTCACC |
| | TACGCCGGCACTGTGCTCCCCCTCAAGGAACTTGCTCTTAAGCACGGTTTCTTCCTGTTCGAGGA |
| | CAGAAAGTTCGCAGATATTGGCAACACTGTCAAGCACCAGTACAAGAACGGTGTCTACCGAATCG |
| | CCGAGTGGTCCGATATCACCAACGCCCACGGTGTACCCGGAACCGGAATCATTGCTGGCCTGCGA |
| | GCTGGTGCCGAGGAAACTGTCTCTGAACAGAAGAAGGAGGACGTCTCTGACTACGAGAACTCCCA |
| | GTACAAGGAGTTCCTGGTCCCCTCTCCCAACGAGAAGCTGGCCAGAGGTCTGCTCATGCTGGCCG |
| | AGCTGTCTTGCAAGGGCTCTCTGGCCACTGGCGAGTACTCCAAGCAGACCATTGAGCTTGCCCGA |
| | TCCGACCCCGAGTTTGTGGTTGGCTTCATTGCCCAGAACCGACCTAAGGGCGACTCTGAGGACTG |
| | GCTTATTCTGACCCCCGGGGTGGGTCTTGACGACAAGGGAGACGCTCTCGGACAGCAGTACCGAA |
| | CTGTTGAGGATGTCATGTCTACCGGAACGGATATCATAATTGTCGGCCGAGGTCTGTACGGCCAG |
| | AACCGAGATCCTATTGAGGAGGCCAAGCGATACCAGAAGGCTGGCTGGGAGGCTTACCAGAAGAT |
| | TAACTGTTAGAGGTTAGACTATGGATATGTAATTTAACTGTGTATATAGAGAGCGTGCAAGTATG |
| | GAGCGCTTGTTCAGCTTGTATGATGGTCAGACGACCTGTCTGATCGAGTATGTATGATACTGCAC |
| | AACCTGTGTATCCGCATGATCTGTCCAATGGGGCATGTTGTTGTGTTTCTCGATACGGAGATGCT |
| | GGGTACAAGTAGCTAATACGATTGAACTACTTATACTTATATGAGGCTTGAAGAAAGCTGACTTG |
| | TGTATGACTTATTCTCAACTACATCCCCAGTCACAATACCACCACTGCACTACCACTACACCTCG |
| | AGCATGCACACTATTATCACATTACTACATCCAAACCCCTACAGGGGGAGGAGCTCTCCAATCAA |
| | ATATGTACATTAACTATCTCTCGTAAATCATTGTTATAAGACCGTCCGTGACTGTCTAAATCGGT |
| | TCATTCGTTGTAACAAATCAGTGTAACAACTCGTGGTACGCCCGGTTTGCTTCGCTCACGCTCCC |
| | CTAGATTTTCCGTCTAGGACACAACAAGCCCTGGTGGATGACGTTCAACGCCTTCAGCGCGTCTT |
| | TTTCCTCAATAACACACGAGATGTTAATTTCGTTGGCTCCCTGAGAAATCATCTCAATGTTAATA |
| | CCTGCCTGGGCCAGAGTAGAGAAGAACATGCCTGCACATCCGACCATGGCCTTCATTCTCGTTCC |
| | GATCAGCGACACAATGGTCATGCCTCGCTTCACATCTACTGTGCCGTACTTTCGCAGCTCCTCCA |
| | CAGCCTGCTTGAGGTTGGAGTCGGGAGCATGGAAGGCCATCGACACATGGACCTCAGAAGTGGAG |
| | ATGAGATCGACGACAAGTTTCTGCTGGTCGAGAGTAGCAAAGATCTTGTTCAAGAAGCCATGACT |
| | CTTGGTTCGCTTATTCGAATGAACGTTGAGAACGGTGATATTCGATTTGGTAGTGACAGCTGTAG |
| | GCTTCTTCTCGTCATCTACAGGAGCAGACACCTGGGGAGTGAATGAGCCAGAGGACAGAGAAGTT |
| | AGCGAGTCGGTGGAAGAAGCCAGATCCTGGACATTGTTTTTCGTTTTCAGGTTGCCCGAGCCGTC |
| | AGCAGATGGGTAGATGATAGTTCCTCCTCCCAGGGGGTTTTCCACGTTTTTGATTCGGATAGGAA |
| | TATGGGCCTTAATGACCTGCTCCATGGTGAAGGGGTGGATGACTTCAGATCCGTAGTAGGTCAAC |
| | TCAGCGGCCTCCTCGGGGGTAATGATGGGCAGCAGACGGGCAGTGGACACCTTTCTGGGATCGGC |
| | AGTGAAGACACCATCGACTTCTTTCCAGATCTGAAGCTCCTTGGCGTCCAGACCCACAGCCACCA |
| | GAGCAGCACACAGATCGGTGTATCCTCGTCCGATCTGGCTTAGGAGGCCTCCCTTGACGGGGCCA |
| | AAGAAACCGGTCAACACGGGCACCATGTTTCCCTTGACAGGAGCAGAGTCCGTGTTAGGAGAGCC |
| | GGGAAGAGTGATGATCTCGCCCAGAACACGTCCGAGGTCCGAATAGAACTTGGGATCTTCGGCAT |
| | TGGTGGTGGTAACTGCGTGAGACAGGTCAAAGTACCGTGCGTTAACACCAGCGTCCCGCATAACT |
| | GCAGTCATGTACATGCAACTGAGCTTCTCCCCAATGGCCATGATGGAGTCGAGTGTTCGGGGGGA |
| | GATCTCAGAAATGATTTCAGCAGCAGCCAGGATTCGCAACAGCTGGTCGCACTCGCCGTTGATGT |
| | CGGCATTGAGGTTTTCGAGCAGTTCGGGGTTCTTGACGTCACGCTTAGCAGCTGCAAGATGGTCT |
| | TCTCGGATAGACTCAATGATGGGGTTGTAGGCGTCTGATCCGAGAAGAGCAGAGTCAGCAGCAGC |
| | AATAAGACGGGTGGTGGTTCCCTCGGCCTTGGTGGCCTCTAGATGGCCTCCTTGGCCCCATTCCA |
| | GCTGCATTAATGAATCGGCCAACGCGCGGGGAGAGGCGGTTTGCGTATTGGGCGCTCTTCCGCTT |
| | CCTCGCTCACTGACTCGCTGCGCTCGGTCGTTCGGCTGCGGCGAGCGGTATCAGCTCACTCAAAG |
| | GCGGTAATACGGTTATCCACAGAATCAGGGGATAACGCAGGAAAGAACATGTGAGCAAAAGGCCA |
| | GCAAAAGGCCAGGAACCGTAAAAAGGCCGCGTTGCTGGCGTTTTTCCATAGGCTCCGCCCCCCTG |
| | ACGAGCATCACAAAAATCGACGCTCAAGTCAGAGGTGGCGAAACCCGACAGGACTATAAAGATAC |
| | CAGGCGTTTCCCCCTGGAAGCTCCCTCGTGCGCTCTCCTGTTCCGACCCTGCCGCTTACCGGATA |
| | CCTGTCCGCCTTTCTCCCTTCGGGAAGCGTGGCGCTTTCTCATAGCTCACGCTGTAGGTATCTCA |
| | GTTCGGTGTAGGTCGTTCGCTCCAAGCTGGGCTGTGTGCACGAACCCCCCGTTCAGCCCGACCGC |
| | TGCGCCTTATCCGGTAACTATCGTCTTGAGTCCAACCCGGTAAGACACGACTTATCGCCACTGGC |
| | AGCAGCCACTGGTAACAGGATTAGCAGAGCGAGGTATGTAGGCGGTGCTACAGAGTTCTTGAAGT |
| | GGTGGCCTAACTACGGCTACACTAGAAGAACAGTATTTGGTATCTGCGCTCTGCTGAAGCCAGTT |
| | ACCTTCGGAAAAAGAGTTGGTAGCTCTTGATCCGGCAAACAAACCACCGCTGGTAGCGGTGGTTT |
| | TTTTGTTTGCAAGCAGCAGATTACGCGCAGAAAAAAAGGATCTCAAGAAGATCCTTTGATCTTTT |
| | CTACGGGGTCTGACGCTCAGTGGAACGAAAACTCACGTTAAGGGATTTTGGTCATGAGATTATCA |
| | AAAAGGATCTTCACCTAGATCCTTTTAAATTAAAAATGAAGTTTTAAATCAATCTAAAGTATATA |
| | TGAGTAAACTTGGTCTGACAGTTACCAATGCTTAATCAGTGAGGCACCTATCTCAGCGATCTGTC |
| | TATTTCGTTCATCCATAGTTGCCTGACTCCCCGTCGTGTAGATAACTACGATACGGGAGGGCTTA |
| | CCATCTGGCCCCAGTGCTGCAATGATACCGCGAGAGCCACGCTCACCGGCTCCAGATTTATCAGC |
| | AATAAACCAGCCAGCCGGAAGGGCCGAGCGCAGAAGTGGTCCTGCAACTTTATCCGCCTCCATCC |
| | AGTCTATTAATTGTTGCCGGGAAGCTAGAGTAAGTAGTTCGCCAGTTAATAGTTTGCGCAACGTT |
| | GTTGCCATTGCTACAGGCATCGTGGTGTCACGCTCGTCGTTTGGTATGGCTTCATTCAGCTCCGG |
| | TTCCCAACGATCAAGGCGAGTTACATGATCCCCCATGTTGTGCAAAAAAGCGGTTAGCTCCTTCG |
| | GTCCTCCGATCGTTGTCAGAAGTAAGTTGGCCGCAGTGTTATCACTCATGGTTATGGCAGCACTG |
| | CATAATTCTCTTACTGTCATGCCATCCGTAAGATGCTTTTCTGTGACTGGTGAGTACTCAACCAA |
| | GTCATTCTGAGAATAGTGTATGCGGCGACCGAGTTGCTCTTGCCCGGCGTCAATACGGGATAATA |
| | CCGCGCCACATAGCAGAACTTTAAAAGTGCTCATCATTGGAAAACGTTCTTCGGGGCGAAAACTC |
| | TCAAGGATCTTACCGCTGTTGAGATCCAGTTCGATGTAACCCACTCGTGCACCCAACTGATCTTC |
| | AGCATCTTTTACTTTCACCAGCGTTTCTGGGTGAGCAAAAACAGGAAGGCAAAATGCCGCAAAAA |
| | AGGGAATAAGGGCGACACGGAAATGTTGAATACTCATACTCTTCCTTTTTCAATATTATTGAAGC |
| | ATTTATCAGGGTTATTGTCTCATGAGCGGATACATATTTGAATGTATTTAGAAAAATAAACAAAT |
| | AGGGGTTCCGCGCACATTTCCCCGAAAAGTGCCACCTGACGTCTAAGAAACCATTATTATCATGA |
| | CATTAACCTATAAAAATAGGCGTATCACGAG |
| 11 | GGTTCCGCTTCCTCGCTCACTGACTCGCTGCGCTCGGTCGTTCGGCTGCGGCGAGCGGTATCAGC |
| | TCACTCAAAGGCGGTAATACGGTTATCCACAGAATCAGGGGATAACGCAGGAAAGAACATGTGAG |
| | CAAAAGGCCAGCAAAAGGCCAGGAACCGTAAAAAGGCCGCGTTGCTGGCGTTTTTCCATAGGCTC |
| | CGCCCCCCTGACGAGCATCACAAAAATCGACGCTCAAGTCAGAGGTGGCGAAACCCGACAGGACT |
| | ATAAAGATACCAGGCGTTTCCCCCTGGAAGCTCCCTCGTGCGCTCTCCTGTTCCGACCCTGCCGC |
| | TTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCA |
| | GCCCGACCGCTGCGCCTTATCCGGTAACTATCGTCTTGAGTCCAACCCGGTAAGACACGACTTAT |
| | CGCCACTGGCAGCAGCCACTGGTAACAGGATTAGCAGAGCGAGGTATGTAGGCGGTGCTACAGAG |
| | TTCTTGAAGTGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCACCGCTGGTA |
| | GCGGTGGTTTTTTTGTTTGCAAGCAGCAGATTACGCGCAGAAAAAAAGGATCTCAAGAAGATCCT |
| | TTGATCTTTTCTACGGGGTCTGACGCTCAGTGGAACGAAAACTCACGTTAAGGGATTTTGGTCAT |
| | GAGATTATCAAAAAGGATCTTCACCTAGATCCTTTTAAATTAAAAATGAAGTTTTAAATCAATCT |
| | AAAGTATATATGAGTAAACTTGGTCTGACAGTTACCAATGCTTAATCAGTGAGGCACCTATCTCA |
| | GCGATCTGTCTATTTCGTTCATCCATAGTTGCCTGACTCCCCGTCGTGTAGATAACTACGATACG |
| | GGAGGGCTTACCATCTGGCCCCAGTGCTGCAATGATACCGCGAGACCCACGCTCACCGGCTCCAG |
| | ATTTATCAGCAATAAACCAGCCAGCCGGAAGGGCCGAGCGCAGAAGTGGTCCTGCAACTTTATCC |
| | GCCTCCATCCAGTCTATTAATTGTTGCCGGGAAGCTAGAGTAAGTAGTTCGCCAGTTAATAGTTT |
| | GCGCAACGTTGTTGCCATTGCTGCAGGCATCGTGGTGTCACGCTCGTCGTTTGGTATGGCTTCAT |
| | TCAGCTCCGGTTCCCAACGATCAAGGCGAGTTACATGATCCCCCATGTTGTGCAAAAAAGCGGTT |
| | AGCTCCTTCGGTCCTCCGATCGTTGTCAGAAGTAAGTTGGCCGCAGTGTTATCACTCATGGTTAT |
| | GGCAGCACTGCATAATTCTCTTACTGTCATGCCATCCGTAAGATGCTTTTCTGTGACTGGTGAGT |
| | ACTCAACCAAGTCATTCTGAGAATAGTGTATGCGGCGACCGAGTTGCTCTTGCCCGGCGTCAACA |
| | CGGGATAATACCGCGCCACATAGCAGAACTTTAAAAGTGCTCATCATTGGAAAACGTTCTTCGGG |
| | GCGAAAACTCTCAAGGATCTTACCGCTGTTGAGATCCAGTTCGATGTAACCCACTCGTGCACCCA |
| | ACTGATCTTCAGCATCTTTTACTTTCACCAGCGTTTCTGGGTGAGCAAAAACAGGAAGGCAAAAT |
| | GCCGCAAAAAAGGGAATAAGGGCGACACGGAAATGTTGAATACTCATACTCTTCCTTTTTCAATA |
| | TTATTGAAGCATTTATCAGGGTTATTGTCTCATGAGCGGATACATATTTGAATGTATTTAGAAAA |
| | ATAAACAAATAGGGGTTCCGCGCACATTTCCCCGAAAAGTGCCACCTGACGTCTAAGAAACCATT |
| | ATTATCATGACATTAACCTATAAAAATAGGCGTATCACGAGGCCCTTTCGTCTGGCCTAGGAAGC |
| | GACTTCCAATCGCTTTGCATATCCAGTACCACACCCACAGGCGTTTGTGCTACTCTACTGATAGC |
| | AATAGATGCGTCATAATTGGTTGGCCCGCTGAGCCTCCACAGGATACTATTGCACATACCCTGGT |
| | CATGTGCAGATCAGCTCATTTGTGGAGACTCTGGAGTAACTTAGACGACGCCTGGTTCAATTGCC |
| | GCAATGTGCGCCCACGCAGATAATGTATTGAGGGGTGGAGCGCCTCTTGGGGACTTGCTGTACTT |
| | GTACGGGATATTAAACGCACTCAGCAAGACCATGACGTAAAACACACCTACTGTACGATACGTAC |
| | TGTAGGTATTGTACTCGTACCCGGTACTACAAATAGTACGATACTATACGGAGTGTATTTGTACC |
| | TTGATATACGACTGGCGGAGTGAAGAGAAGGAGTTGAACAAGACCAGATGGGGATATCAGCCCCA |
| | GTGCTTTGTATTACAAGTACGAGTACTTAATAGATACTGTAAGGCTATTGATACGGATGGCAGTA |
| | AGTCATTGAGTAAGCAATTGTGGCCCAGCATCTCCCCTACGTACTTGTACCATACCCCATGGAGA |
| | CACCAATGGTCTTTCACGCACACTGTCGTGTGCTGTATCGCAGAATCGGGTGTCCAACCAAATGC |
| | CGTTACCCCCACGTCACAGCCGATAGACAGATACACCATCAATACCAGCAGGTTGTATCATGCGG |
| | TTGGCTGAAGGTAAGCTGATTGGTCTAAAAACTGTAGCTGTCCTAATTCAACGAGCGCTATTTGG |
| | GGCCAACCACCTCGGCCAAGCGGCCTTTAATCTGCGTGCCCCAGAGGCGTCTAATGAGGCTCTGG |
| | CCGCCACTGTAGGAGTGTTTCTCTGTGCGCACACGCAGTTTTGAGTTTGGGCGACTTTCCCTTTT |
| | TCCCAATTGCGTACACACACAGCTCCGAGCTAAGCGCTGTCCTTGAACCTTCTCCCTCTTTTCCC |
| | TCTTTTTCTCTTCCCCTTCCCCTCCTCCACATTAAGGCCAAATCCTGAATTGCACCAACTAGTAC |
| | AACGACAACAATGGACAAGAAGTACTCCATCGGTTTGGACATTGGTACTAACTCTGTCGGCTGGG |
| | CCGTCATCACCGACGAGTACAAGGTTCCCTCCAAGAAGTTCAAGGTCCTTGGCAACACCGACCGA |
| | CACTCTATCAAGAAGAACCTGATCGGTGCTCTGCTGTTCGACTCTGGCGAGACTGCCGAGGCCAC |
| | CCGACTGAAGCGAACCGCTCGACGCCGATACACCCGACGAAAGAACCGAATCTGTTACCTCCAGG |
| | AGATCTTCAGCAACGAGATGGCTAAGGTCGACGACTCCTTCTTCCACCGACTCGAGGAGTCTTTC |
| | CTGGTCGAAGAGGATAAGAAGCACGAGCGACACCCCATCTTCGGCAACATTGTTGATGAGGTTGC |
| | CTACCATGAGAAGTACCCCACCATCTACCACCTCCGAAAGAAGCTCGTCGACTCCACTGACAAGG |
| | CTGACCTCCGACTCATCTACCTTGCTCTCGCCCACATGATCAAGTTCCGAGGTCACTTCCTCATT |
| | GAGGGTGATCTCAACCCCGACAACTCCGACGTTGACAAGCTGTTCATCCAGCTCGTCCAGACCTA |
| | CAACCAGCTCTTTGAGGAGAACCCTATCAACGCTTCTGGTGTTGACGCCAAGGCCATTCTCTCCG |
| | CCCGACTCTCTAAGTCCCGACGACTCGAGAACCTCATTGCCCAGCTGCCCGGCGAGAAGAAGAAC |
| | GGCCTCTTCGGTAACCTGATTGCTCTCTCTCTTGGTCTGACCCCCAACTTCAAGTCCAACTTTGA |
| | CCTCGCCGAGGACGCCAAGCTCCAGCTGTCCAAGGACACCTACGATGACGATCTGGACAACCTCC |
| | TGGCCCAGATCGGTGACCAGTACGCCGATCTCTTCCTTGCCGCCAAGAACCTCTCCGACGCCATC |
| | CTGCTCTCCGACATCCTCCGAGTCAACACCGAGATTACCAAGGCTCCTCTGTCTGCCTCTATGAT |
| | CAAGCGATACGACGAGCACCACCAGGATCTCACTCTTCTCAAGGCTCTCGTCCGACAGCAGCTCC |
| | CCGAGAAGTACAAGGAGATTTTCTTTGACCAGTCCAAGAACGGTTACGCTGGCTACATTGACGGT |
| | GGTGCTTCCCAGGAAGAGTTTTACAAGTTCATCAAGCCTATTCTGGAGAAGATGGACGGTACCGA |
| | GGAGCTGCTCGTCAAGCTCAACCGAGAGGACCTCCTTCGAAAGCAGCGAACCTTCGATAACGGCT |
| | CCATCCCCCACCAGATCCACCTGGGTGAGCTCCACGCCATTCTCCGAAGACAAGAGGACTTCTAC |
| | CCCTTCCTAAAGGATAACCGAGAGAAGATCGAGAAGATTCTCACCTTCCGAATCCCCTACTACGT |
| | CGGTCCCCTCGCTCGAGGTAACTCCCGATTTGCTTGGATGACCCGAAAGTCCGAGGAGACTATCA |
| | CCCCCTGGAACTTTGAAGAGGTAGTCGACAAGGGTGCCTCCGCCCAGTCTTTCATTGAGCGGATG |
| | ACCAACTTCGATAAGAACCTCCCCAACGAGAAGGTCCTTCCCAAGCACTCTCTCCTCTACGAGTA |
| | CTTCACCGTCTACAACGAGCTGACCAAGGTCAAGTACGTTACCGAGGGCATGCGAAAGCCCGCTT |
| | TCCTCTCTGGTGAGCAGAAGAAGGCCATTGTCGACCTCCTGTTCAAGACTAACCGAAAAGTCACC |
| | GTCAAGCAGCTCAAGGAAGACTACTTCAAGAAGATTGAGTGCTTCGACTCCGTCGAGATTTCCGG |
| | TGTCGAGGACCGATTCAACGCCTCCCTCGGCACCTACCACGATCTTCTGAAGATCATCAAGGACA |
| | AGGACTTTCTTGATAACGAGGAGAACGAGGACATTCTCGAGGACATCGTCCTCACCCTCACCCTT |
| | TTCGAGGATCGAGAGATGATCGAGGAGCGACTCAAGACCTACGCCCATCTCTTCGACGACAAGGT |
| | CATGAAGCAACTCAAGCGACGACGATACACTGGCTGGGGCCGACTTTCCCGAAAGCTCATCAACG |
| | GCATCCGAGACAAGCAGTCTGGCAAGACCATCCTGGACTTCCTGAAGTCCGACGGTTTCGCCAAC |
| | CGAAACTTCATGCAGCTCATCCACGACGACTCTCTTACCTTCAAAGAGGATATCCAGAAGGCCCA |
| | GGTTTCTGGCCAGGGCGACTCCCTCCACGAGCACATTGCCAACCTCGCCGGATCCCCCGCCATCA |
| | AAAAGGGTATCCTCCAGACCGTCAAGGTTGTCGACGAACTCGTGAAGGTCATGGGCCGACACAAG |
| | CCCGAGAACATCGTTATCGAGATGGCCCGAGAGAACCAGACCACCCAGAAGGGTCAGAAGAACTC |
| | CCGAGAGCGAATGAAGCGAATCGAAGAGGGTATCAAGGAGCTCGGTTCCCAGATTCTCAAGGAGC |
| | ACCCCGTCGAGAACACCCAGCTCCAGAACGAGAAACTCTACCTGTACTACCTCCAGAATGGCCGA |
| | GACATGTACGTTGACCAGGAGCTCGACATCAACCGACTCTCCGACTACGACGTCGACCACATTGT |
| | TCCTCAGTCCTTCCTCAAGGACGACTCCATCGACAACAAGGTTCTGACCCGATCTGACAAGAACC |
| | GAGGTAAGTCCGACAACGTTCCCTCCGAAGAGGTCGTTAAGAAGATGAAGAACTACTGGCGACAG |
| | CTTCTCAACGCCAAACTGATCACCCAGCGAAAGTTTGACAACCTCACCAAGGCCGAGCGAGGTGG |
| | TCTGTCCGAGCTGGACAAGGCCGGCTTCATTAAGCGACAGCTGGTCGAGACTCGACAGATCACCA |
| | AGCACGTCGCCCAGATCCTCGACTCCCGAATGAACACCAAGTACGACGAGAACGACAAGCTCATC |
| | CGGGAGGTCAAGGTCATCACCCTGAAGTCTAAGCTTGTCTCCGACTTCCGAAAGGACTTCCAGTT |
| | CTACAAGGTCCGAGAGATCAACAACTACCACCACGCCCACGACGCCTACCTCAACGCCGTTGTTG |
| | GTACCGCCCTCATCAAGAAGTATCCCAAGCTCGAGTCCGAGTTCGTTTACGGCGACTACAAGGTT |
| | TACGATGTCCGAAAGATGATTGCCAAGTCCGAGCAGGAGATCGGTAAGGCCACCGCCAAGTACTT |
| | TTTCTACTCCAACATCATGAATTTCTTCAAGACCGAGATCACTCTCGCCAACGGTGAGATTCGAA |
| | AGCGACCCCTGATTGAGACTAATGGTGAGACTGGTGAGATCGTCTGGGATAAGGGCCGAGACTTC |
| | GCCACCGTCCGAAAGGTCCTGTCCATGCCCCAGGTCAACATTGTCAAGAAGACCGAGGTCCAGAC |
| | CGGTGGCTTCTCCAAGGAGTCCATTCTCCCCAAGCGAAACTCCGACAAACTCATCGCCCGTAAGA |
| | AGGACTGGGATCCGAAGAAGTACGGTGGTTTCGATTCTCCCACCGTTGCCTACTCCGTCCTCGTT |
| | GTTGCTAAAGTCGAGAAGGGTAAGTCTAAGAAACTCAAGTCCGTGAAGGAGCTACTCGGTATCAC |
| | CATCATGGAGCGATCTTCTTTTGAGAAGAACCCCATTGACTTCCTCGAGGCCAAGGGTTACAAAG |
| | AGGTCAAGAAGGACCTGATTATCAAGCTGCCCAAGTACTCCCTCTTTGAGCTCGAGAACGGCCGA |
| | AAGCGAATGCTGGCTTCCGCTGGTGAGCTGCAGAAGGGCAACGAGCTCGCTCTGCCCTCCAAGTA |
| | CGTCAACTTCCTCTACCTGGCCTCCCACTACGAGAAGCTCAAGGGCTCCCCCGAGGACAACGAGC |
| | AGAAGCAGCTGTTCGTTGAGCAGCACAAGCACTACCTCGACGAGATCATCGAGCAGATCTCCGAG |
| | TTCTCCAAGCGAGTCATCCTCGCTGACGCCAACCTTGATAAGGTTCTCTCTGCTTACAACAAGCA |
| | CCGGGACAAGCCCATCCGAGAGCAGGCCGAGAATATCATCCACCTCTTCACTCTCACCAACCTCG |
| | GCGCTCCTGCTGCCTTCAAGTACTTCGACACCACCATTGACCGAAAGAGGTACACCTCCACCAAG |
| | GAAGTCCTCGACGCCACCCTGATCCACCAGTCCATCACCGGCCTCTACGAAACCCGAATCGACCT |
| | CTCCCAGCTCGGCGGTGACTCTCGAGCCGACCCCAAGAAGAAGCGAAAAGTCTAAATATCCGAAG |
| | ATCAAGAGCGAAGCAAGTTGTAAGTCCAGGACATGTTTCCCGCCCACGCGAGTGATTTATAACAC |
| | CTCTCTTTTTTGACACCCGCTCGCCTTGAAATTCATGTCACATAAATTATAGTCAACGACGTTTG |
| | AATAACTTGTCTTGTAGTTCGATGATGATCATATGATTACATTAATAGTAATTACTGTATTTGAT |
| | ATATATACTAATTACAATAGTACATATTAGAACATACAATAGTTAGTGCCGTGAAGTGGCTTAAA |
| | ATACCGCGAGTCGATTACGTAATATTATTACCTCTTGCCCATCGAACGTACAAGTACTCCTCTGT |
| | TCTCTCCTTCCTTTGCTTTGTGCACGAAGAACTGCGGTCAGGTGACACAACTTTTTCCATCTCAG |
| | GGTGTGTCGCGTGTGCTTCATCCAAACTTTAGTTGGGGTTCGGGTTCGCGCGAGATGATCACGTG |
| | CCCTGATTTGGTGTCGTCCCCCGTCGCGCTGCGCACGTGATTTATTTATTTCCGGTGGCTGCTGT |
| | CTACGCGGGGCCTTCTCTGCCCTTCTGTTTCAACCTTCGGGCGGTTCTCGTAACCAGCAGTAGCA |
| | ATCCATTTCGAAACTCAAAGAGCTAAAAACGTTAAACCTCAGCAGTCGCTCGACGAATGGGCTGC |
| | GGTTGGGAAGCCCACGAGGCCTATAGCCAGAGCCTCGAGTTGACAGGAGCCCAGACGCCTTTTCC |
| | AACGGCAACTTTTATATAAAATGGCAATGTATTCATGCAATTGCGGCCGTGTCAGGTTGGAGACA |
| | CTGGACCACACTCTCCATTGCTTCCTGAGGAGATGGATCATTGCTAGTGCATCTACGCGCAGCAA |
| | TCCCGCAAGCTCGACAACCGTAGATGGGCTTTGGTGGGCCAATCAATTACGCAACCCGCACGTTA |
| | AATTGTATGAGGAAGGAAGGCCACGGTACAAAGTGGGTGGTCTTCACCCAGTGGTTGTTGGTGGC |
| | GTCATGCAGACCATGCATTGGGGATAGCACAGGGTTGGGGTGTCTTGTGGACTCAATGGGTGAAA |
| | GGAGATGGAAAAGGGCGGTGAAAAGTGGTAGAATCGAAATCCCTGACGTCAATTTATAAAGTAAA |
| | ATGCGTTTCTGCCATTTTGCTCCCCTCCTTCTTTCGCAATCGCCTCCCCAAAAGTTGTCGTGGCA |
| | GTACACATGCTTGCATACAATGAAGCTAATCCGGCTTGCTCAGTAGTTGCTATATCCAGGCATGG |
| | TGTGAAACCCCTCAAAGTATATATAGGAGCGGTGAGCCCCAGTCTGGGGTCTTTTCTCTCCATCT |
| | CAAAACTACTTTCTCACAATGACGAGACTGATGAGTCCGTGAGGACGAAACGAGTAAGCTCGTCG |
| | AGGGCTGCAAAATTCACGAGTTTTAGAGCTAGAAATAGCAAGTTAAAATAAGGCTAGTCCGTTAT |
| | CAACTTGAAAAAGTGGCACCGAGTCGGTGCTTTTGGCCGGCATGGTCCCAGCCTCCTCGCTGGCG |
| | CCGGCTGGGCAACATGCTTCGGCATGGCGAATGGGACTAAACTTCGAGCTAATCCAGTAGCTTAC |
| | GTTACCCAGGGGCAGGTCAACTGGCTAGCCACGAGTCTGTCCCAGGTCGCAATTTAGTGTAATAA |
| | ACAATATATATATTGAGTCTAAAGGGAATTGTAGCTATTGTGATTGTGTGATTTTCGTCTTGCTG |
| | GTTCTTATTGTGTCCCATTCGTTTCATCCTGATGAGGACCCCTGGAACCGGTGTTTTCTTAGTCT |
| | CTGCAATCGCTAGTCTTGTTGCTATGACAGTTGCGTCGACACTATTCAGGTCATCTATCGGTTAT |
| | TCTGATATTATAATACCTCCGGATCGATGTACCTGATTTATACTTGCAGCAATGTTTACTTCTTA |
| | TCGTTGGACCCCGTCTTCAATTACACTTCCCAACTGGGAACACCCCTCTTTATCGACCCATTTTA |
| | GGTAATTTACCCTAGCCCATTGTCTCCATAAGGAATATTACCCTAACCCACAGTCCAGGGTGCCC |
| | AGGTCCTTCTTTGGCCAAATTTTAACTTCGGTCCTATGGCACAGCGGTAGCGCGTGAGATTGCAA |
| | ATCTTAAGGTCCCGAGTTCGAATCTCGGTGGGACCTAGTTGAAAAATACCTCTAATGCGCCGATG |
| | GTTTAGTGGTAAAATCCATCGTTGCCATCGATGGGCCCCCGGTTCGATTCCGGGTCGGCGCAGGT |
| | TGACGTACAGCAGGCTGAACGAGGATGTTTTAGAGCTAGAAATAGCAAGTTAAAATAAGGCTAGT |
| | CCGTTATCAACTTGAAAAAGTGGCACCGAGTCGGTGCTTTTTTTTTTCGCGATACACGAATGTGA |
| | TACGGATCAAAGTAAGCAGGACTACGATAAGATAACGAATGCGGTGCAGTCCATGTCGATTAGGT |
| | ATAGATACATTTATTTTGTGTTATGTTACATTTTGGGGGGATACTGTCCTACTTGTAGTACCTAC |
| | TTGTAGTGGCGCGTCTATTCCTTTGCCCTCGGACGAGTGCTGGGGCGTCGGTTTCCACTATCGGC |
| | GAGTACTTCTACACAGCCATCGGTCCAGACGGCCGCGCTTCTGCGGGCGATTTGTGTACGCCCGA |
| | CAGTCCCGGCTCCGGATCGGACGATTGCGTCGCATCGACCCTGCGCCCAAGCTGCATCATCGAAA |
| | TTGCCGTCAACCAAGCTCTGATAGAGTTGGTCAAGACCAATGCGGAGCATATACGCCCGGAGCCG |
| | CGGCGATCCTGCAAGCTCCGGATGCCTCCGCTCGAAGTAGCGCGTCTGCTGCTCCATACAAGCCA |
| | ACCACGGCCTCCAGAAGAAGATGTTGGCGACCTCGTATTGGGAATCCCCGAACATCGCCTCGCTC |
| | CAGTCAATGACCGCTGTTATGCGGCCATTGTCCGTCAGGACATTGTTGGAGCCGAAATCCGCGTG |
| | CACGAGGTGCCGGACTTCGGGGCAGTCCTCGGCCCAAAGCATCAGCTCATCGAGAGCCTGCGCGA |
| | CGGACGCACTGACGGTGTCGTCCATCACAGTTTGCCAGTGATACACATGGGGATCAGCAATCGCG |
| | CATATGAAATCACGCCATGTAGTGTATTGACCGATTCCTTGCGGTCCGAATGGGCCGAACCCGCT |
| | CGTCTGGCTAAGATCGGCCGCAGCGATCGCATCCATGGCCTCCGCGACCGGCTGCAGAACAGCGG |
| | GCAGTTCGGTTTCAGGCAGGTCTTGCAACGTGACACCCTGTGCACGGCGGGAGATGCAATAGGTC |
| | AGGCTCTCGCTGAATTCCCCAATGTCAAGCACTTCCGGAATCGGGAGCGCGGCCGATGCAAAGTG |
| | CCGATAAACATAACGATCTTTGTAGAAACCATCGGCGCAGCTATTTACCCGCAGGACATATCCAC |
| | GCCCTCCTACATCGAAGCTGAAAGCACGAGATTCTTCGCCCTCCGAGAGCTGCATCAGGTCGGAG |
| | ACGCTGTCGAACTTTTCGATCAGAAACTTCTCGACAGACGTCGCGGTGAGTTCAGGCTTTTTCAT |
| | ATGGGTACCTGAGAACATTTTTGTGTCTAGGTGTTTGTGTTTGGACTGCGATCAGTGAAGAAAAG |
| | AAGAGGAAAAATTGTGCAAGAAATTTTGCTTTCAAGACTTGGCTGATGCAGCAGGGTAACTCTGG |
| | GACACAGACCTATGTTTGTGGTTAAACTCAATGCACGTGGTACGTGCGTGGAGCGCTTACCCATC |
| | CAAGGGTGTGGACATGGAACCGACGGTCCGTGGAGTTGTGTAATGTCATTTTGGCGACTCTTGAA |
| | GCAAGGCTATAAAAAAATTGTGTGGCTTGAGTCTTATCGAGCTCGGTCACTACAAGAGTTAATCT |
| | TCCTGTCTCAGGCAGACAGGTCAGGCAGGGTTACTTTTGGGTGTGCTGTAACTCACTGTATGGCC |
| | GTTAGTGCGCATAGACGTTGTACATACTGGACCGAATTGTAGCGTGCTCAATAGGGCCAATAAAG |
| | CTATTGTAGGGATCCGAATTTTCAGAACCTAATTTATCTGTTACCCGGCCTGTGGCTCGCACAGC |
| | TTAAAAATGGTCAAACTTTCCCCTTCTTGTCTTTTTTTCCTCACATTCATCAGGTTCTTGTCTTG |
| | ATCTTTCAAGTGAGTATTAATTACCGACCTTGGTTCTTCATTGGGAGAGCATTGGAAGCCGTGGT |
| | GCAGCAACCACAAAACGGTTCTTCCCCTTCGATACCTTCTTGCCTGCCTTTCAATACAAGTCGGC |
| | TCGATTAGCGGTGGTCGCCCCCGCCAGCGGAGAACATGGAACTAACCCAGAATGAGAGCTAAGTG |
| | GAGAAAGAAGAGAGTCAGACGACTCAAGCGAAAGCGCCGCAAGGTCCGAGCTCGATCCAAATAAG |
| | CGGTTTTTAACGGAGATTTAACACTAAATCGAAGAACTTTTCCCGTTTCATTTGCGAATGAGCTC |
| | GTTAACAAAATCCCCCAGTTTTTTTATCCAGCTGTAAGGATTGACATTAGTAATGAATTATTGTT |
| | TGGTATATTTAAATCTGTAGTTCCTTTCTGTCCGTGTCGGCAACTGTCGTACTCGTGATTTACTT |
| | GTATTGACGAATACTTACTGTAGCGCACTCTGCTGCTACTGGTCGTAAGGATGTGCTATTTCGGT |
| | GTATGGTGGGTTTTTTGGGGGTCGGAACCGAAGACTGTTACACGGGCACGGCTCGTTGTGTACAC |
| | GCACAGAGCTCTTGCGAGTCATGTTGTAGCTAGCTCGTCGTGTTCAGGAACTGTTCGATGGTTCG |
| | GAGAGAGTCGCCGCCCAGAACATACGCGCACCGATGTCAGCAGACAGCCTTATTACAAGTATATT |
| | CAAGCAAGTATATCCGTAGGGTGCGGGTGATTTGGATCTAAGGTTCGTACTCAACACTCACGAGC |
| | AGCTTGCCTATGTTACATCCTTTTATCAGACATAACATAATTGGAGTTTACTTACACACGGGGTG |
| | TACCTGTATGAGCACCACCTACAATTGTAGCACTGGTACTTGTACAAAGAATTTATTCGTACGAA |
| | TCACAGGGACGGCCGCCCTCACCGAACCAGCGAATACCTCAGCGGTCCCCTGCAGTGACTCAACA |
| | AAGCGATATGAACATCTTGCGATGGTATCCTGCTGATAGTTTTTACTGTACAAACACCTGTGTAG |
| | CTCCTTCTAGCATTTTTAAGTTATTCACACCTCAAGGGGAGGGATAAATTAAATAAATTCCAAAA |
| | GCGAAGATCGAGAAACTAAATTAAAATTCCAAAAACGAAGTTGGAACACAACCCCCCGAAAAAAA |
| | ACAACAAACAAAAAACCCAACAAAATAAACAAAAACAAAATAAATATATAACTACCAGTATCTGA |
| | CTAAAAGTTCAAATACTCGTACTTACAACAAATAGAAATGAGCCGGCCAAAATTCTGCAGAAAAA |
| | AATTTCAAACAAGTACTGGTATAATTAAATTAAAAAACACATCAAAGTATCATAACGTTAGTTAT |
| | TTTATTTTATTTAATAAAAGAAAACAACAAGATGGGCTCAAAACTTTCAACTTATACGATACATA |
| | CCAAATAACAATTTAGTATTTATCTAAGTGCTTTTCGTAGATAATGGAATACAAATGGATATCCA |
| | GAGTATACACATGGATAGTATACACTGACACGACAATTCTGTATCTCTTTATGTTAACTACTGTG |
| | AGGCATTAAATAGAGCTTGATATATAAAATGTTACATTTCACAGTCTGAACTTTTGCAGATTACC |
| | TAATTTGGTAAGATATTAATTATGAACTGAAAGTTGATGGCATCCCTAAATTTGATGAAAGATGA |
| | AATTGTAAATGAGGTGGTAAAAGAGCTACAGTCGTTTTGTTTTGAGATACCATCATCTCTAACGA |
| | AATATCTATTAAAAATCTCAGTGTGATCATGAGTCATTGCCATCCTGGAAAATGTCATCATGGCT |
| | GATATTTCTAACTGTTTACTTGAGATAAATATATATTTACAAGAACTTCCCTTGAAATTAATTTA |
| | GATATAAAATGTTTGCGGGCAAGTTACTACGAGGAATAAATTATATCTAGA |
| 12 | MGDLDARGTSAHPELSERPSIMPSMSDIQDPSGDDKATPRGSAAGLPQLELAGHARRLG |
| | HLENFFAVQHRQQIYSSFAVFCEFDTACSLAQLASAVRNVCLSNPLLLHTVEPKHPDIA |
| | GFYHSDEYLSRPWPQHDYMRVLREVHVADVVMNGQKEHAHVVRDAVDVFQAHGNQVTSE |
| | LLELMTQIEIPHASQTRPSWRLLCFPHGEANRWRTFAFVSNHCSSDGLSGLNFERDLQK |
| | ELAHGPTSGAPGAPGASGVIFDYAQDAATLPKLPPPIDQKLDYRPSKKALLGLLAGKFV |
| | REKLGYVSAAPPTTPTSDLAHPEGHQYYCYLVNVPTSSVAHIKTQVRENVPHKCTLTPF |
| | LQACWLVSLFKYGRVESGSWLERYTDVLVAMNTRQLLPEDLELQRQYRYGSNVGGVRYN |
| | YPIAPLDVRDNDQKFWSLVESYRLALSDARDKNDYLYALGALMLPEIYEKKNVDAVVND |
| | TILNQRRSGTLISNVGYVRDEQPTAFAIKNHVFSQGVGANRNAFVLNICATDQGGLNIA |
| | ISIAKGTLASRQEGQELCDIFKSTLLRF |

1. Process for in situ extraction of retinoids in a fermentation process, comprising cultivation of the host cell, particularly fungal host cell, preferably oleaginous yeast, more preferably Yarrowia, in the presence of a second phase lipophilic solvent comprising vegetable oil.

2. Process according to claim 1, wherein the host cell is grown on a carbon source selected from glucose or ethanol.

3. Process according to claim 1, wherein the percentage of retinoids is increased by at least about 10% compared to a process using silicone oil as second phase lipophilic solvent.

4. Process according to claim 1, wherein the solvent is used for collection and extraction of the fermentative produced retinoids.

5. Process according to claim 1, wherein the fermentative produced retinoids comprise a percentage of at least about 0.001 to about 30% retinyl acetate based on total retinoids.

6. Process according to claim 1, wherein the vegetable oil is selected from the group consisting of oleic, palmitic, steric or linoleic acid and glycerol, preferably selected from the group consisting of corn oil, olive oil, cottonseed oil, rapeseed oil, sesame oil, canola oil, safflower oil, sunflower oil, soybean oil, grapeseed oil, and peanut oil, more preferably selected from corn oil.

7. Process according to claim 1, wherein the host cell is selected from oleaginous host cell including Rhodosporidium, Lipomyces or Yarrowia, preferably Yarrowia lipolytica.

8. Process according to claim 1, wherein the host cell comprises one or more genetic modifications, particularly reduction or abolishment of endogenous lipase activity, preferably reduction or abolishment of lipase activity corresponding to Yarrowia lipase activity 2, 3, 4, and/or 8, more preferably Yarrowia lipase activity 8.

9. Process according to claim 1, wherein the host cell is transformed with and is expressing heterologous genes, preferably genes encoding acetylating enzymes catalyzing the conversion of retinol to retinyl acetate, more preferably fungal enzymes, most preferably originated from Lachancea mirantina.

10. Process according to claim 9, wherein the acetylating enzyme comprises one or more amino acid substitution(s) in a sequence with at least about 20%, such as e.g. 25, 30, 35, 40, 45, 50, 55, 60, 65, 70, 75, 80, 85, 90, 92, 95, 97, 98, 99% or up to 100% identity to SEQ ID NO:12, wherein the one or more amino acid substitution(s) are located at position(s) corresponding to amino acid residue(s) selected from the group consisting of position 68, 69, 72, 73, 171, 174, 176, 178, 291, 292, 294, 301, 307, 308, 311, 312, 320, 322, 334, 362, 405, 407, 409, 480, 483, 484, 490, 492, 520, 521, 522, 524, 525, 526 and combinations thereof in a polypeptide according to SEQ ID NO:12.
